(19) United States
(12) Patent Application Publication
(10) Pub. No.: US 2006/0094923 A1
(43) Pub. Date: May 4, 2006

(54) Systems and methods for treating a patient using radiation therapy

(75) Inventor: Timothy P. Mate, Bellevue, WA (US)

Correspondence Address: PERKINS COIE LLP;PATENT-SEA, P.O. BOX 1247, SEATTLE, WA, 98111-1247, US

(73) Assignee: Calypso Medical Technologies, Inc., Seattle, WA (US)

(21) Appl. No.: 11/243,478

(22) Filed: Oct. 3, 2005

Related U.S. Application Data
(60) Provisional application No. 60/615,443, filed on Oct. 1, 2004.

(51) Int. Cl. A61N 5/00

(52) U.S. Cl. 600/3; 600/7

(57) ABSTRACT

Apparatus and methods for treating a patient using radiation therapy. In one embodiment, an apparatus comprises a tube configured to receive a radiation source and an expandable member. The tube has a first end configured to be inserted into a patient and a second end that is generally configured to remain external to the patient. The expandable member is at the first end of the tube, and it is configured to contain the radiation source. The expandable member can comprise a balloon, flexible bladder, mechanical linkage (e.g., a cage), a mesh, or other suitable expandable systems. The apparatus further includes a marker associated with the expandable member such that the marker moves with the expandable member.

### CROSS REFERENCE TO RELATED APPLICATIONS

This application claims the benefit of U.S. Provisional Application No. 60/615,443 filed on Oct. 1, 2004, which is herein incorporated by reference in its entirety.

### TECHNICAL FIELD

This invention relates generally to systems and method for accurately locating, tracking or otherwise monitoring a device or target for treating a patient using radiation.

### BACKGROUND OF THE INVENTION

Cancer is a disease that begins in the cells of the patient. Radiation therapy has become a significant and highly successful process for treating breast cancer, lung cancer, brain cancer and many other types of localized cancers. Radiation therapy is particularly useful for treating tissue after a tumor has been removed, centrally located tumors, and/or small cell tumors that cannot be removed surgically. Radiation therapy can be used as a curative treatment or as a palliative treatment when a cure is not possible. Additionally, surgery and chemotherapy can be used in combination with radiation therapy.

Breast cancer has recently been treated by surgically removing cancerous breast tissue and subsequently treating the remaining tissue surrounding the lumpectomy cavity using radiation. Proxima Corporation and Xoft, Inc. have developed devices and systems for treating the breast tissue surrounding the cavity created by a lumpectomy. The existing breast brachytherapy devices have a balloon configured to be implanted in the breast and a radiation source that can be placed within the balloon. After performing a lumpectomy, the balloon is inserted into the surgical cavity and inflated until the balloon presses against the tissue. The balloon is typically left in the patient for approximately five days over which two radiation treatments per day are performed. Each radiation treatment includes inserting the radiation source into the balloon and activating the radiation source to deliver ionizing radiation for approximately 10-15 minutes. After all of the radiation treatments have been performed during the multi-day course of treatment, the balloon is deflated and removed from the patient.

One challenge of these procedures is inflating the balloon to a desired size and monitoring the balloon to ensure that the balloon has maintained the desired size throughout the multi-day course of treatment. The size of the balloon is currently determined by instilling radiopaque contrast into the balloon and measuring a resulting CT or X-ray image using a ruler. The patient must accordingly undergo a CT scan or another type of X-ray to obtain the image, and then a practitioner must evaluate the image to determine if the balloon is at the desired size. This is a time-consuming and expensive process that reduces the efficiency of treating the patients, and it should be performed each day during the course of treatment. This process also exposes the patient to additional radiation. Therefore, there is a need to provide accurate measurements of the size of the balloon throughout the course of treatment.

Another challenge of existing breast brachytherapy systems is assessing the relative position and/or rotational orientation of the balloon within the lumpectomy cavity. The balloon may move within the lumpectomy cavity over the course of treatment, but existing systems do not detect the relative position between the balloon and the breast. Moreover, when the radiation source is asymmetrically positioned within the balloon (e.g., spaced apart from a rotational center line of the balloon), it is important to know the rotational orientation of the balloon within the lumpectomy cavity so that the radiation source is located at a desired distance from the tissue. Conventional techniques that use a radiopaque contrast in the balloon do not identify the relative position or rotational orientation of the balloon. This can be problematic because the balloon can move after it has been implanted over the course of treatment, or the balloon may not inflate as planned. Therefore, it would also be desirable to determine the rotational orientation or other relative movement of the balloon within the cavity.

### BRIEF DESCRIPTION OF THE DRAWINGS

FIG. 1 is an isometric view schematically illustrating a stage of a lumpectomy procedure to remove a cancerous tumor from a patient.

FIG. 2 is an isometric view illustrating a later stage of the lumpectomy procedure.

FIG. 3 is an isometric view of an apparatus for facilitating radiation treatment of a target in accordance with an embodiment of the invention.

FIG. 4 is an isometric view illustrating an implementation of an apparatus for radiation treatment in accordance with an embodiment of the invention.

FIG. 5 is a side elevation view of a tracking system for localizing and monitoring an apparatus in accordance with an embodiment of the invention.

FIG. 6 is a schematic elevation view of a patient on a support using an apparatus for facilitating radiation treatment of a target in accordance with an embodiment of the invention.

FIG. 7 is a side view schematically illustrating the operation of a localization system for use with an apparatus for facilitating radiation treatment in accordance with an embodiment of the invention.

FIG. 8 is a schematic view further illustrating the operation of an apparatus for facilitating radiation treatment of a target in accordance with an embodiment of the invention.

FIG. 9 is a schematic, side cross-sectional view of an apparatus for radiation treatment in accordance with an embodiment of the invention.

FIG. 10 is a schematic, side cross-sectional view of an apparatus for facilitating radiation treatment in accordance with another embodiment of the invention.

FIG. 11 is a schematic, side cross-sectional view of another apparatus for facilitating radiation treatment of a target in accordance with an embodiment of the invention.

FIG. 12A is an isometric view of a marker for use with a localization system in accordance with an embodiment of the invention.

FIG. 12B is a cross-sectional view of the marker of FIG. 12A taken along line 12B-12B.

FIG. 12C is an illustration of a radiographic image of the marker of FIGS. 12A-B.

FIG. 13A is an isometric view of a marker for use with a localization system in accordance with another embodiment of the invention.

FIG. 13B is a cross-sectional view of the marker of FIG. 13A taken along line 13B-13B.

FIG. 14A is an isometric view of a marker for use with a localization system in accordance with another embodiment of the invention.

FIG. 14B is a cross-sectional view of the marker of FIG. 14A taken along line 14B-14B.

FIG. 15 is an isometric view of a marker for use with a localization system in accordance with another embodiment of the invention.

FIG. 16 is an isometric view of a marker for use with a localization system in accordance with yet another embodiment of the invention.

FIG. 17 is a schematic block diagram of a localization system for use in tracking a target in accordance with an embodiment of the invention.

FIG. 18 is a schematic view of an array of coplanar source coils carrying electrical signals in a first combination of phases to generate a first excitation field.

FIG. 19 is a schematic view of an array of coplanar source coils carrying electrical signals in a second combination of phases to generate a second excitation field.

FIG. 20 is a schematic view of an array of coplanar source coils carrying electrical signals in a third combination of phases to generate a third excitation field.

FIG. 21 is a schematic view of an array of coplanar source coils illustrating a magnetic excitation field for energizing markers in a first spatial orientation.

FIG. 22 is a schematic view of an array of coplanar source coils illustrating a magnetic excitation field for energizing markers in a second spatial orientation.

FIG. 23A is an exploded isometric view showing individual components of a sensor assembly for use with a localization system in accordance with an embodiment of the invention.

FIG. 23B is a top plan view of a sensing unit for use in the sensor assembly of FIG. 23A.

FIG. 24 is a schematic diagram of a preamplifier for use with the sensor assembly of FIG. 23A.

FIG. 25 is a graph of illustrative tumor motion ellipses from experimental phantom based studies of the system.

FIG. 26 is a graph of root mean square (RMS) error from experimental phantom based studies of the system.

FIG. 27 is an exemplary histogram of localization error from experimental phantom based studies of the system.

FIG. 28 is graph of position error as a function of speed from experimental phantom based studies of the system.

In the drawings, identical reference numbers identify similar elements or components. The sizes and relative positions of elements in the drawings are not necessarily drawn to scale. For example, the shapes of various elements and angles are not drawn to scale, and some of these elements are arbitrarily enlarged and positioned to improve drawing legibility. Further, the particular shapes of the elements as drawn, are not intended to convey any information regarding the actual shape of the particular elements, but rather the shapes have been solely selected for ease of recognition in the drawings.

### DETAILED DESCRIPTION

In the following description, certain specific details are set forth in the context of breast brachytherapy in order to provide a thorough understanding of various embodiments of the invention. However, one skilled in the relevant art will recognize that the invention may be practiced without one or more of these specific details, or with other methods, components, materials, etc. For instance, inflatable devices for temporary or permanent implantation in a patient can have one or more markers as described below for use in beam radiation therapy procedures described in U.S. patent application Ser. No. 11/165,843, filed on 24 Jun. 2005, and Ser. No. 11/166,801, filed on 24 Jun. 2005, both of which are incorporated herein by reference. In other instances, well-known structures associated with target locating and tracking systems have not been shown or described in detail to avoid unnecessarily obscuring descriptions of the embodiments of the invention.

Unless the context requires otherwise, throughout the specification and claims which follow, the word “comprise” and variations thereof, such as, “comprises” and “comprising” are to be construed in an open, inclusive sense that is as “including, but not limited to.” Reference throughout this specification to “one embodiment” or “an embodiment” means that a particular feature, structure or characteristic described in connection with the embodiment is included in at least one embodiment of the present invention. Thus, the appearances of the phrases “in one embodiment” or “in an embodiment” in various places throughout this specification are not necessarily all referring to the same embodiment. Further more, the particular features, structures, or characteristics may be combined in any suitable manner in one or more embodiments. The headings provided herein are for convenience only and do not interpret the scope or meaning of the claimed invention.

A. Overview

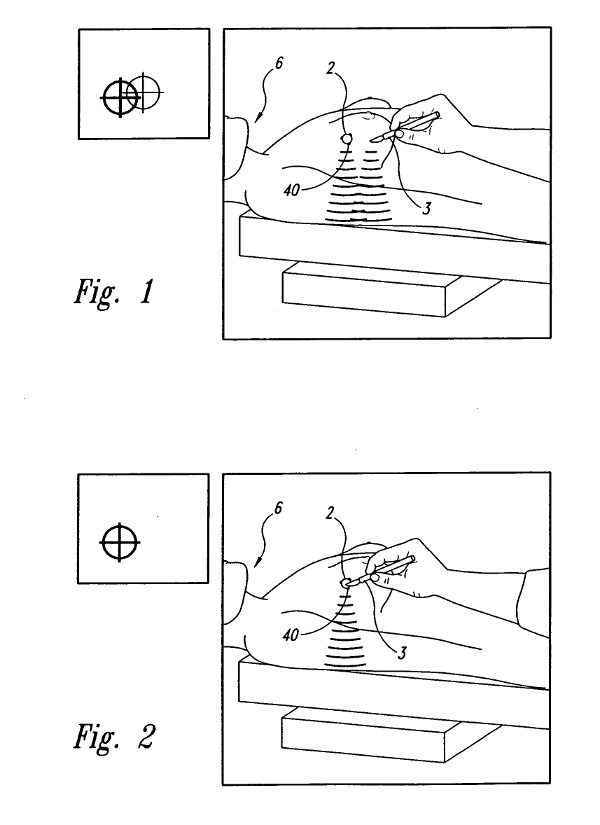

FIGS. 1-28 illustrate a system and several components for locating, tracking and monitoring a target within a patient in accordance with embodiments of the present invention. The system and components guide or otherwise monitor radiation therapy to more effectively treat the target. Several of the components described below with reference to FIGS. 1-28 can also be used to treat targets in other parts of the body in accordance with other aspects of the present invention. Additionally, like reference numbers refer to like components and features throughout the various figures.

One aspect of the invention is directed toward apparatus for facilitating radiation treatment of a target in a patient. One embodiment of such an apparatus comprises a tube configured to receive a radiation source and an expandable member. The tube has a first end configured to be inserted into a patient and a second end that is generally configured to remain external to the patient. The expandable member is at the first end of the tube, and it is configured to contain the radiation source. The expandable member can comprise a balloon, flexible bladder, mechanical linkage (e.g., a cage), a mesh, or other suitable expandable systems. The apparatus further includes a marker associated with the expandable member such that the marker moves with the expandable member. The marker generally comprises a wireless transponder configured to wirelessly transmit a location signal in response to a wirelessly transmitted excitation energy, but in other embodiments the marker can be a radiopaque element. One suitable marker comprises a casing and a magnetic transponder having a coil in a capacitor coupled to the coil.

The marker is associated with the expandable member such that the marker moves with the expandable member to an expanded orientation. In one embodiment, the marker is attached to or otherwise embedded in the wall of the expandable member. In other embodiments, the marker can be attached to a sheath or mesh around the expandable member.

Another aspect of the invention is directed toward methods for facilitating radiation treatment of a target in a patient. One embodiment of such a method comprises positioning an expandable member in the patient with respect to the target, and expanding the expandable member to a desired size within the patient. This method further includes determining a parameter of the expandable member by localizing a marker that moves in association with the expandable member. This method can optionally include inserting an ionizing radiation source into the expandable member and delivering ionizing radiation to the target. This method can further optionally include localizing the position of the ionizing radiation source within the expandable member by localizing a marker attached to the ionizing radiation source.

Before treating the target with radiation, a portion of the tumor may be surgically removed from the patient. In the case of treating breast cancer, for example, a patient undergoes a lumpectomy to remove as much of the tumor as possible while minimizing removal of healthy tissue. FIGS. 1 and 2 illustrate performing a lumpectomy using guided surgical techniques as disclosed in U.S. Pat. No. 6,918,919, owned by Calypso Medical Technologies, which is incorporated herein by reference. As shown in FIG. 1, a marker 40 is implanted within or at least proximate to a target 2 of a patient 6, and a marker (not shown) is attached to a scalpel 3. The location of the target 2 is determined by tracking the marker 40 using a localization system that wirelessly operates with the marker 40. Referring to FIG. 2, the localization system correlates the location of the marker 40 and the scalpel 3 or other tool so that the surgeon can accurately remove as much of the target 2 as possible. Although the tracking system for enhancing lumpectomies is very useful, cancerous breast tissue may remain in the breast. As such, breast brachytherapy has been developed to further treat the tissue proximate to the lumpectomy cavity.

Various embodiments of the invention are described in this section to provide specific details for a thorough understanding and enabling description of these embodiments. A person skilled in the art, however, will understand that the invention may be practiced without several of these details, or that additional details can be added to the invention. Where context permits, singular or plural terms may also include the plural or singular term, respectively. Moreover, unless the word “or” is expressly limited to mean only a single item exclusive from other items in reference to a list of at least two items, then the use of “or” in such a list is to be interpreted as including (a) any single item in the list, (b) all of the items in the list, or (c) any combination of the items in the list.

B. Embodiments of Apparatus for Facilitating Radiation Treatment

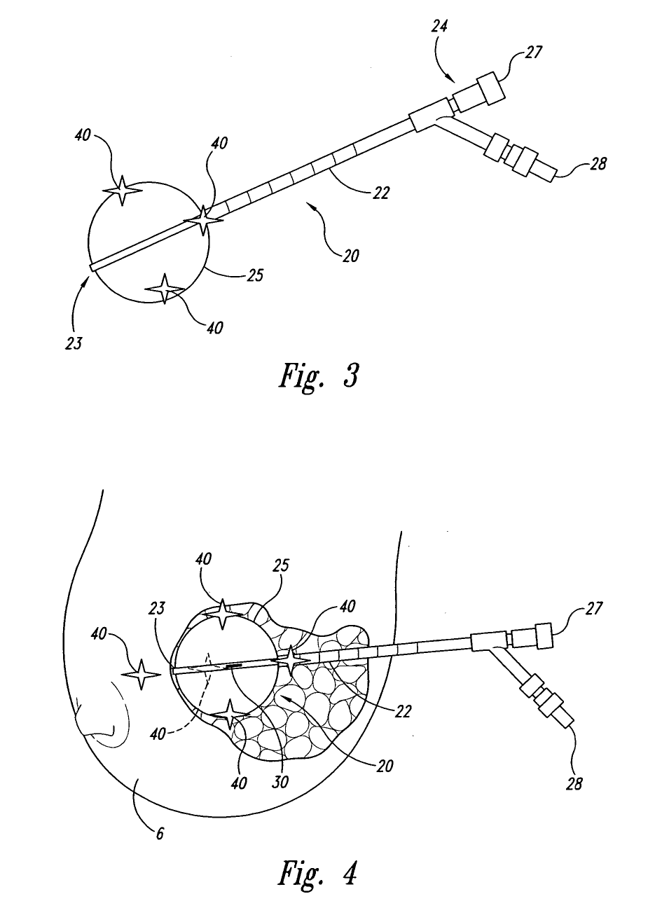

FIG. 3 is an isometric view of an apparatus 20 for facilitating radiation treatment of a target in accordance with an embodiment of the invention. The apparatus includes a tube 22 having a first end 23 configured to be inserted into the patient and a second end 24. The tube 22 can be a catheter, such as a multilumen silicon catheter, or other type of device that can be percutaneously inserted into the breast or other part of the body. The apparatus 20 further includes an expandable member 25 at the first end 23 of the tube 22. The expandable member 25 can be a balloon or other type of device configured to create and hold a desired shape. For example, the expandable member 25 can be an inflatable bladder, mechanical linkage, a mesh of shape memory material, or other suitable devices that can be inserted into a patient and moved between a collapsed configuration and an enlarged or expanded configuration. When the expandable member 25 is a balloon, it is typically inserted into the patient in a collapsed configuration (not shown) and then filled with a saline solution until the balloon expands to a desired diameter. The apparatus 20 further includes a first port 27 and a second port 28. As explained in more detail below, a radiation source is inserted through the first port 27 until the radiation source is positioned within the balloon 25. Additionally, the saline solution or other type of solution is passed through the second port 28 to expand/contract the expandable member 25.

The apparatus 20 further includes a plurality of markers 40 that are associated with the expandable member 25 such that the markers 40 move with the expandable member 25 to an expanded orientation. In several embodiments, the markers 40 comprise wireless transponders configured to wirelessly transmit independent location signals in response to wirelessly transmitted excitation energies. For example, the markers can comprise a casing and a magnetic transponder in the casing as described in more detail below. In several embodiments, the markers 40 are attached to or otherwise embedded in the expandable member 25 such that the markers move in direct correspondence to the movement of the expandable member 25. In other embodiments, the markers 40 are attached to a sheath or a mesh that surrounds the expandable member 25 so that expansion of the expandable member 25 causes a corresponding expansion of the sheath or mesh. In either case, the markers 40 are associated with the expandable member 25 such that the markers 40 move with the expandable member 25 at least when the expandable member 25 approaches the expanded orientation.

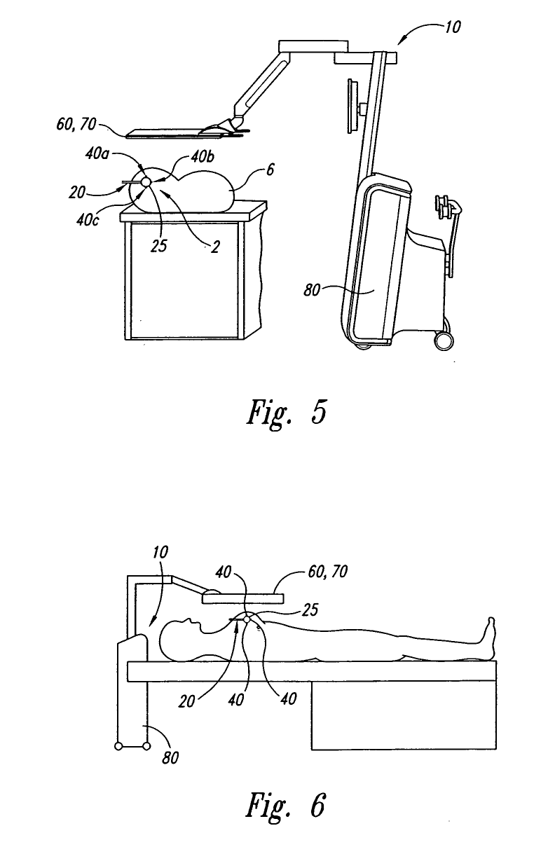

FIG. 4 is an isometric view schematically illustrating an implementation of the apparatus 20 for performing a breast brachytherapy procedure. The tube 20 is inserted into the breast of the patient 6 when the expandable member 25 is in a contracted or collapsed configuration (not shown in FIG. 4). As the first end 23 passes through the breast, the markers 40 can be tracked using a localization system, such as the localization systems described below with reference to FIGS. 5-28. When the apparatus 20 is at a desired location relative to a target within the breast, saline is injected through the second port 28 to inflate the expandable member 25 within the lumpectomy cavity. The expandable member 25 is inflated until it reaches a desired diameter to position a radiation source at a desired distance from the tissue. A radiation source 30 is then inserted through the first port 27 and into the expandable member 25. The radiation source 30 can be a suitable radiation source manufactured by Proxima Corporation or Xoft, Inc. as disclosed in U.S. Pat. Nos. 6,200,257; 6,083,148; 6,022,308; 5,931,774; 5,913,813; 6,537,195; 6,390,967; 6,319,188; and U.S. Publication No. US2005/0124844A1, all of which are incorporated herein by reference. A source marker 40 (shown in broken line) can be associated with the radiation source 30 such that this marker moves with the radiation source 30 as it is positioned within the expandable member 25. The radiation source 30 is then activated to deliver radiation to the target tissues surrounding the expandable member 25.

The apparatus 20 provides several advantages for performing breast brachytherapy. In several embodiments, the apparatus 20 enables an accurate determination of the size of the expandable member inflated within the breast without taking expensive CT images and manually assessing the images. This aspect is very useful because the diameter or size of the expandable member 25 positions the radiation source 30 at a desired distance from the tissue to deliver a more uniform and penetrating dose of radiation, but the expandable member 25 may change over the course of the treatment. For example, the expandable member 25 may collapse or have a slow leak such that the size and shape of the expandable member may change over the multi-day treatment course. By localizing the relative positions of the markers 40, any changes in the size and shape of the expandable member 25 can be determined before, during, and after each treatment session to ensure that the accurate dose of radiation is delivered from the radiation source 30.

Another advantage of the apparatus 20 is that movement of the expandable member 25 relative to the breast can be determined. In additional embodiments, a separate marker 40 is implanted or otherwise attached to the patient 6 to define a known reference location. The reference marker 40 can be attached to the surface of the breast, or in other applications it is attached to a fixed structure of the patient (e.g., chest wall, etc.). By localizing the reference marker 40 and the markers 40 associated with the expandable member 25, relative movement of the expandable member 25 within the breast can be determined throughout the course of therapy to ensure that the expandable member is positioned at the desired location within the patient. This is also useful for detecting movement of the patient during therapy. As a result, the apparatus 20 is expected to provide accurate measurements to confirm the status and the location of the expandable member throughout the course of therapy.

Another advantage of the apparatus 20 is that the rotational orientation of the expandable member 25 relative to the breast can be determined and assessed throughout the course of treatment. As mentioned above, the rotational orientation of the expandable member 25 may be particularly important in applications in which the radiation source 30 is located asymmetrically relative to the expandable member 25. The markers 40 can be tracked or otherwise located using a localization system to determine the rotational orientation of the expandable member 25, and thus determine the position of the radiation source 30 relative to the tissue. Therefore, the apparatus 20 is expected to be particularly useful in cases that use asymmetric radiation sources.

FIGS. 5 and 6 illustrate a localization system 10 for determining the actual location of the markers 40 in a three-dimensional reference frame when the markers are within or on the patient 6. In the illustrated embodiment shown in FIGS. 5 and 6, more specifically, three markers identified individually as markers 40a-c are associated with the expandable member 25 of the apparatus 20. In other applications, a single marker, two markers, or more than three markers can be used depending upon the particular application. Two markers, for example, are highly desirable because the target can be located accurately, and also because relative displacement between the markers over time can be used to monitor the status and position of the expandable member 25 in the patient 6. In a particular embodiment of the system illustrated in FIGS. 5 and 6, the localization system 10 tracks the three-dimensional coordinates of the markers 40a-c in real time to an absolute external reference frame during the setup process and while irradiating the patient to mitigate collateral effects on adjacent healthy tissue and to ensure that the desired dosage is applied to the target tissue.

1. General Operation of Selected Markers and Localization Systems

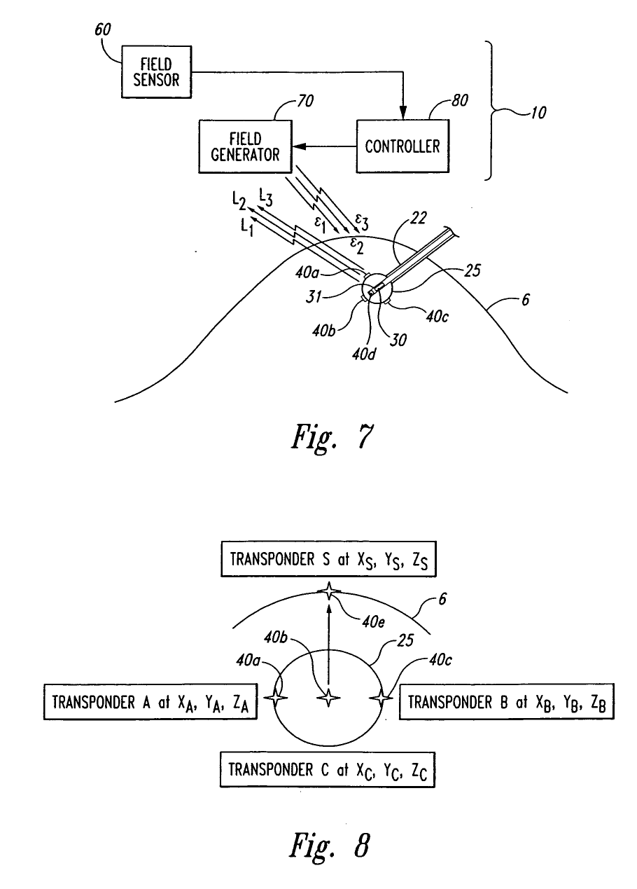

FIG. 7 is a schematic view illustrating the operation of an embodiment of the localization system 10 and markers 40a-c for treating a target in the breast of the patient. The markers 40a-c are used to determine the size and location of the expandable member 25, and a marker 40d can be used to determine the position of the radiation source 30 or a catheter 31 before, during and after radiation sessions. More specifically, the localization system 10 determines the locations of the markers 40a-c and provides objective target position data to a memory, user interface, linear accelerator and/or other device in real time during setup, treatment, deployment, simulation, surgery, and/or other medical procedures. In one embodiment of the localization system, real time means that indicia of objective coordinates are provided to a user interface at (a) a sufficiently high refresh rate (i.e., frequency) such that pauses in the data are not humanly discernable and (b) a sufficiently low latency to be at least substantially contemporaneous with the measurement of the original signal. In other embodiments, real time is defined by higher frequency ranges and lower latency ranges for providing the objective data, or in still other embodiments, real time is defined as providing objective data responsive to the location of the markers (e.g., at a periodicity or frequency that adequately tracks the location of the target in real time and/or at a latency that is at least substantially contemporaneous with obtaining position data of the markers).

The localization system 10 includes an excitation source 60 (e.g., a pulsed magnetic field generator), a sensor assembly 70, and a controller 80 coupled to both the excitation source 60 and the sensor assembly 70. The excitation source 60 generates an excitation energy to energize at least one of the markers 40a-c in the patient 6 (FIG. 5). The embodiment of the excitation source 60 shown in FIG. 7 produces a pulsed magnetic field at different frequencies. For example, the excitation source 60 can frequency multiplex the magnetic field at a first frequency E₁ to energize the first marker 40a, a second frequency E₂ to energize the second marker 40b, and a third frequency E₃ to energize the third marker 40c. In response to the excitation energy, the markers 40a-c generate location signals L₁₋₃ at unique response frequencies. More specifically, the first marker 40a generates a first location signal L₁ at a first frequency in response to the excitation energy at the first frequency E₁, the second marker 40b generates a second location signal L₂ at a second frequency in response to the excitation energy at the second frequency E₂, and the third marker 40c generates a third location signal L₃ at a third frequency in response to the excitation energy at the third frequency E₃. In an alternative embodiment with two markers, the excitation source generates the magnetic field at frequencies E₁ and E₂, and the markers 40a-b generate location signals L₁ and L₂, respectively.

The sensor assembly 70 can include a plurality of coils to sense the location signals L₁₋₃ from the markers 40a-c. The sensor assembly 70 can be a flat panel having a plurality of coils that are at least substantially coplanar relative to each other. In other embodiments, the sensor assembly 70 may be a non-planar array of coils.

The controller 80 includes hardware, software or other computer-operable media containing instructions that operate the excitation source 60 to multiplex the excitation energy at the different frequencies E₁₋₃. For example, the controller 80 causes the excitation source 60 to generate the excitation energy at the first frequency E₁ for a first excitation period, and then the controller 80 causes the excitation source 60 to terminate the excitation energy at the first frequency E₁ for a first sensing phase during which the sensor assembly 70 senses the first location signal L₁ from the first marker 40a without the presence of the excitation energy at the first frequency E₁. The controller 80 then causes the excitation source 60 to: (a) generate the second excitation energy at the second frequency E₂ for a second excitation period; and (b) terminate the excitation energy at the second frequency E₂ for a second sensing phase during which the sensor assembly 70 senses the second location signal L₂ from the second marker 40b without the presence of the second excitation energy at the second frequency E₂. The controller 80 then repeats this operation with the third excitation energy at the third frequency E₃ such that the third marker 40c transmits the third location signal L₃ to the sensor assembly 70 during a third sensing phase. As such, the excitation source 60 wirelessly transmits the excitation energy in the form of pulsed magnetic fields at the resonant frequencies of the markers 40a-c during excitation periods, and the markers 40a-c wirelessly transmit the location signals L₁₋₃ to the sensor assembly 70 during sensing phases. It will be appreciated that the excitation and sensing phases can be repeated to permit averaging of the sensed signals to reduce noise.

The computer-operable media in the controller 80, or in a separate signal processor, also includes instructions to determine the absolute positions of each of the markers 40a-c in a three-dimensional reference frame. Based on signals provided by the sensor assembly 70 that correspond to the magnitude of each of the location signals L₁₋₃, the controller 80 and/or a separate signal processor calculates the absolute coordinates of each of the markers 40a-c in the three-dimensional reference frame.

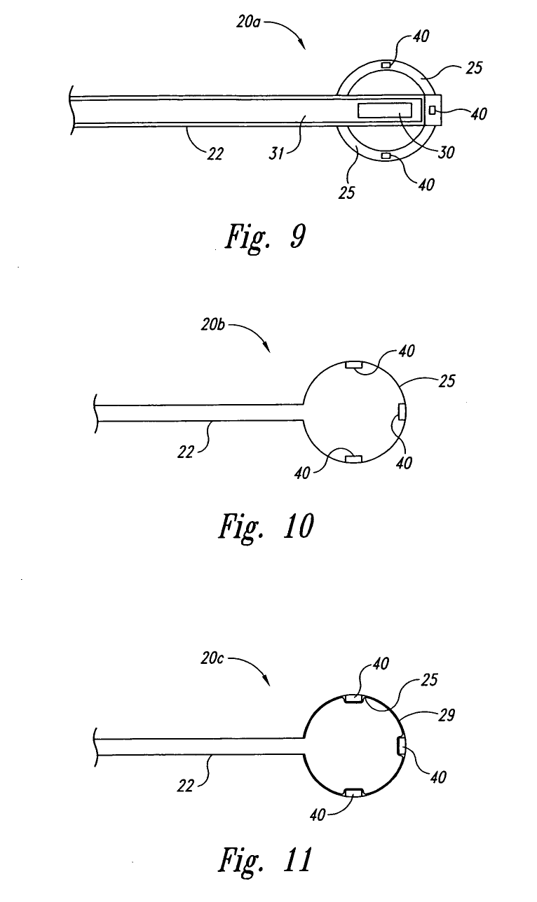

FIG. 8 schematically illustrates the location of the markers that are obtained by the localization system 10. In this embodiment, markers 40a-c are associated with the expandable member 25 and another marker 40e is a reference marker attached to the patient 6. The localization system 10 provides three-dimensional coordinates for each of the transponders in real time to determine a parameter of the expandable member 25 and/or the location of a radiation source within the expandable member 25. In the particular embodiment shown in FIG. 8, the localization system 10 determines a coordinate for the first marker 40 (X_{A}, Y_{A}, Z_{A}), along with corresponding coordinates for the markers 40b, 40c and 40e. Based upon the coordinates of the markers 40a-c, the size, shape, and rotational orientation of the expandable member 25 within the patient 6 can be readily determined by relative changes between these coordinates. Additionally, based upon the coordinates of the markers 40a-c and the reference marker 40e, the relative position of the expandable member 25 within the patient can be determined throughout the course of treatment.

2. Real Time Tracking

The localization system 10 and markers 40 enable real time tracking of the target 2, expandable member 25, and/or the radiation source 30 relative to an external reference frame outside of the patient during treatment planning, set up, irradiation sessions, and at other times of the radiation therapy process. In many embodiments, real time tracking means collecting position data of the markers, determining the locations of the markers in an external reference frame (i.e., a reference frame outside the patient), and providing an objective output in the external reference frame responsive to the location of the marker. The objective output is provided at a frequency/periodicity that adequately tracks the target in real time, and/or a latency that is at least substantially contemporaneous with collecting the position data (e.g., within a generally concurrent period of time).

For example, several embodiments of real time tracking are defined as determining the locations of the markers and calculating the locations relative to an external reference frame at (a) a sufficiently high frequency/periodicity so that pauses in representations of the target location at a user interface do not interrupt the procedure or are readily discernable by a human, and (b) a sufficiently low latency to be at least substantially contemporaneous with the measurement of the location signals from the markers. Alternatively, real time means that the location system 10 calculates the absolute position of each individual marker 40 and/or the location of the target at a periodicity of approximately 1 ms to 5 seconds, or in many applications at a periodicity of approximately 10-100 ms, or in some specific applications at a periodicity of approximately 20-50 ms. In applications for user interfaces, for example, the periodicity can be 12.5 ms (i.e., a frequency of 80 Hz), 16.667 ms (60 Hz), 20 ms (50 Hz), and/or 50 ms (20 Hz). Additionally, real time tracking can further mean that the location system 10 provides the absolute locations of the markers 40, the target 2, the expandable member 25 and/or the radiation source 30 to a memory device, user interface, linear accelerator or other device within a latency of 10 ms to 5 seconds from the time the localization signals were transmitted from the markers 40. In more specific applications, the location system generally provides the locations of the markers 40, target 2, or an instrument within a latency of about 20-50 ms. The location system 10 accordingly provides real time tracking to monitor the position of the markers 40 and/or the target 2 with respect to an external reference frame in a manner that is expected to enhance the efficacy of radiation therapy.

Alternatively, real time tracking can further mean that the location system 10 provides the absolute locations of the markers 40 and/or the target 2 to a memory device, user interface or other device within a latency of 10 ms to 5 seconds from the time the localization signals were transmitted from the markers 40. In more specific applications, the location system generally provides the locations of the markers 40 and/or target 2 within a latency of about 20-50 ms. The location system 10 accordingly provides real time tracking to monitor the position of the markers 40 and/or the target 2 with respect to an external reference frame in a manner that is expected to enhance the efficacy of radiation therapy because higher radiation doses can be applied to the target and collateral effects to healthy tissue can be mitigated.

Alternatively, real-time tracking can further be defined by the tracking error. Measurements of the position of a moving target are subject to motion-induced error, generally referred to as a tracking error. According to aspects of the present invention, the localization system 10 and at least one marker 4 enable real time tracking of the target 2 or other instrument relative to an external reference frame with a tracking error that is within clinically meaningful limits.

Tracking errors are due to two limitations exhibited by any practical measurement system, specifically (a) latency between the time the target position is sensed and the time the position measurement is made available, and (b) sampling delay due to the periodicity of measurements. For example, if a target is moving at 5 cm/s and a measurement system has a latency of 200 ms, then position measurements will be in error by 1 cm. The error in this example is due to latency alone independent of any other measurement errors, and is simply due to the fact that the target or instrument has moved between the time its position is sensed and the time the position measurement is made available for use. If this exemplary measurement system further has a sampling periodicity of 200 ms (i.e., a sampling frequency of 5 Hz), then the peak tracking error increases to 2 cm, with an average tracking error of 1.5 cm.

For a real time tracking system to be useful in medical applications, it is desirable to keep the tracking error within clinically meaningful limits. For example, in a system for tracking motion of a tumor or an instrument for radiation therapy, it may be desirable to keep the tracking error within 5 mm. Acceptable tracking errors may be smaller when tracking other organs for radiation therapy. In accordance with aspects of the present invention, real time tracking refers to measurement of target position and/or rotation with tracking errors that are within clinically meaningful limits.

3. Additional Embodiments of Apparatus for Facilitating Radiation Treatment

FIGS. 9-11 illustrate additional embodiments of apparatus for facilitating radiation treatment of a target in a patient. FIG. 9, more specifically, illustrates an apparatus 20a that includes the tube 22 and the expandable member 25 at one end of the tube 22. The markers 40 can be embedded within the wall of the expandable member 25 such that the markers 40 move with the expandable member 25 as it is inflated and deflated. The apparatus 20a can include one or more markers 40 embedded within wall of the expandable member 25, and in optional embodiments a marker 40 can also be attached to a distal end of the tube 22. In operation, a radiation source 30 attached to a shaft or catheter 31 is passed through the tube 22 until the radiation source 30 is positioned at a desired location within the expandable member 25. As explained above, another marker can be attached to the catheter 31.

FIG. 10 is a schematic cross-sectional view illustrating an apparatus 20b in accordance with yet another embodiment of the invention. In this embodiment, the apparatus includes the shaft 22 and the expandable member 25 at one end of the shaft. The markers 40 are attached to an interior or exterior surface of the expandable member 25. The markers can be adhered or otherwise attached to the desired surface of the expandable member 25 using an adhesive.

FIG. 11 illustrates another embodiment of an apparatus 20c in accordance with the invention. In this embodiment, the apparatus 20c includes the tube 22 and the expandable member 25 attached to one end of the tube. This embodiment can further include a flexible member 29 around or otherwise attached to the expandable member 25. The markers 40 are attached to the flexible member 29, and the flexible member 29 can be a sheath or a mesh. In operation, the expandable member 25 presses against the flexible member 29 and expands the flexible member 29 to fill the lumpectomy cavity in the patient. The markers 40 accordingly travel with the movement of the expandable member 25.

C. Specific Embodiments of Markers and Localization Systems

The following specific embodiments of markers, excitation sources, sensors and controllers provide additional details to implement the systems and processes described above with reference to FIGS. 1-11. The present inventors overcame many challenges to develop markers and localization systems that accurately determine the location of a marker which (a) produces a wirelessly transmitted location signal in response to a wirelessly transmitted excitation energy, and (b) has a cross-section small enough to be implanted in a patient. Systems with these characteristics have several practical advantages, including (a) not requiring ionization radiation, (b) not requiring line-of-sight between the markers and sensors, and (c) effecting an objective measurement of the location and/or rotation of an instrument or target. The following specific embodiments are described in sufficient detail to enable a person skilled in the art to make and use such a localization system for radiation therapy involving the breast of the patient, but the invention is not limited to the following embodiments of markers, excitation sources, sensor assemblies and/or controllers.

1. Markers

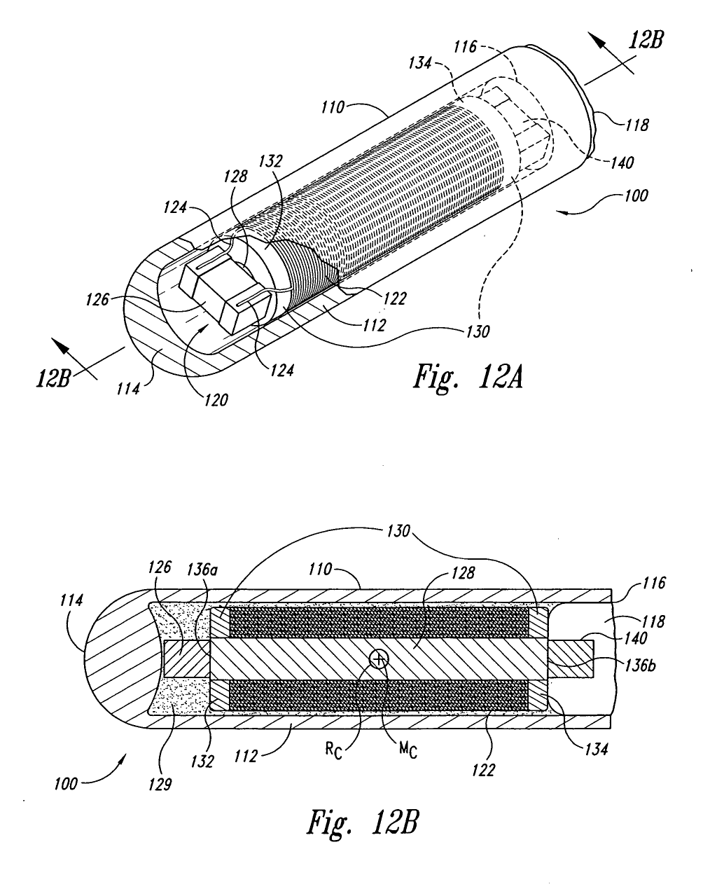

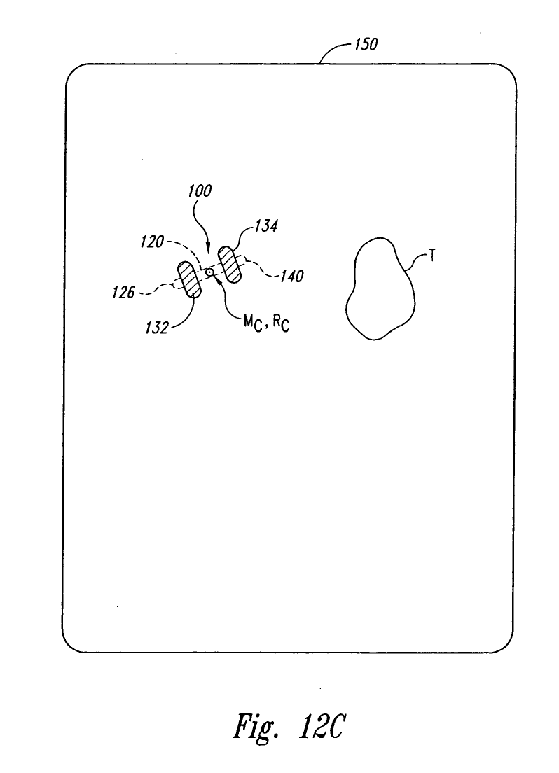

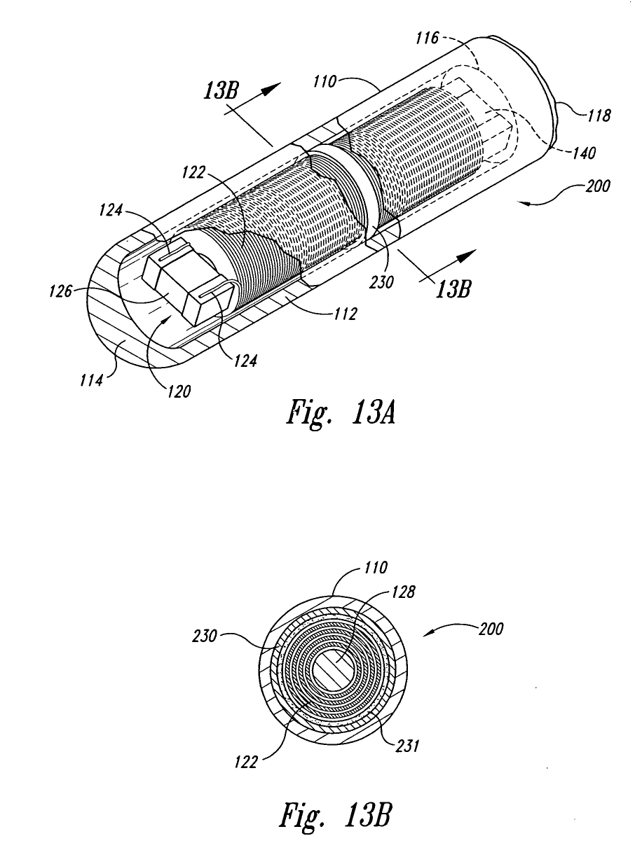

FIG. 12A is an isometric view of a marker 100 for use with the localization system 10 (FIGS. 1-7). The embodiment of the marker 100 shown in FIG. 12A includes a casing 110 and a magnetic transponder 120 (e.g., a resonating circuit) in the casing 110. The casing 110 is a barrier configured to be implanted in the patient, or encased within the body of an instrument. The casing 110 can alternatively be configured to be adhered externally to the skin of the patient. The casing 110 can be a generally cylindrical capsule that is sized to fit within the bore of a small introducer, such as bronchoscope or percutaneous trans-thoracic implanter, but the casing 110 can have other configurations and be larger or smaller. The casing 110, for example, can have barbs or other features to anchor the casing 110 in soft tissue or an adhesive for attaching the casing 110 externally to the skin of a patient. Suitable anchoring mechanisms for securing the marker 100 to a patient are disclosed in International Publication No. WO 02/39917 A1, which designates the United States and is incorporated herein by reference. In one embodiment, the casing 110 includes (a) a capsule or shell 112 having a closed end 114 and an open end 116, and (b) a sealant 118 in the open end 116 of the shell 112. The casing 110 and the sealant 118 can be made from plastics, ceramics, glass or other suitable biocompatible materials.

The magnetic transponder 120 can include a resonating circuit that wirelessly transmits a location signal in response to a wirelessly transmitted excitation field as described above. In this embodiment, the magnetic transponder 120 comprises a coil 122 defined by a plurality of windings of a conductor 124. Many embodiments of the magnetic transponder 120 also include a capacitor 126 coupled to the coil 122. The coil 122 resonates at a selected resonant frequency. The coil 122 can resonate at a resonant frequency solely using the parasitic capacitance of the windings without having a capacitor, or the resonant frequency can be produced using the combination of the coil 122 and the capacitor 126. The coil 122 accordingly generates an alternating magnetic field at the selected resonant frequency in response to the excitation energy either by itself or in combination with the capacitor 126. The conductor 124 of the illustrated embodiment can be hot air or alcohol bonded wire having a gauge of approximately 45-52. The coil 122 can have 800-1000 turns, and the windings are preferably wound in a tightly layered coil. The magnetic transponder 120 can further include a core 128 composed of a material having a suitable magnetic permeability. For example, the core 128 can be a ferromagnetic element composed of ferrite or another material. The magnetic transponder 120 can be secured to the casing 110 by an adhesive 129.

The marker 100 also includes an imaging element that enhances the radiographic image of the marker to make the marker more discernible in radiographic images. The imaging element also has a radiographic profile in a radiographic image such that the marker has a radiographic centroid at least approximately coincident with the magnetic centroid of the magnetic transponder 120. As explained in more detail below, the radiographic and magnetic centroids do not need to be exactly coincident with each other, but rather can be within an acceptable range.

FIG. 12B is a cross-sectional view of the marker 100 along line 12B-12B of FIG. 12A that illustrates an imaging element 130 in accordance with an embodiment of the invention. The imaging element 130 illustrated in FIGS. 12A-B includes a first contrast element 132 and second contrast element 134. The first and second contrast elements 132 and 134 are generally configured with respect to the magnetic transponder 120 so that the marker 100 has a radiographic centroid R_{c} that is at least substantially coincident with the magnetic centroid M_{c} of the magnetic transponder 120. For example, when the imaging element 130 includes two contrast elements, the contrast elements can be arranged symmetrically with respect to the magnetic transponder 120 and/or each other. The contrast elements can also be radiographically distinct from the magnetic transponder 120. In such an embodiment, the symmetrical arrangement of distinct contrast elements enhances the ability to accurately determine the radiographic centroid of the marker 100 in a radiographic image.

The first and second contrast elements 132 and 134 illustrated in FIGS. 12A-B are continuous rings positioned at opposing ends of the core 128. The first contrast element 132 can be at or around a first end 136a of the core 128, and the second contrast element 134 can be at or around a second end 136b of the core 128. The continuous rings shown in FIGS. 12A-B have substantially the same diameter and thickness. The first and second contrast elements 132 and 134, however, can have other configurations and/or be in other locations relative to the core 128 in other embodiments. For example, the first and second contrast elements 132 and 134 can be rings with different diameters and/or thicknesses.

The radiographic centroid of the image produced by the imaging element 130 does not need to be absolutely coincident with the magnetic centroid M_{c}, but rather the radiographic centroid and the magnetic centroid should be within an acceptable range. For example, the radiographic centroid R_{c} can be considered to be at least approximately coincident with the magnetic centroid M_{c} when the offset between the centroids is less than approximately 5 mm. In more stringent applications, the magnetic centroid M_{c} and the radiographic centroid R_{c} are considered to be at least substantially coincident with each other when the offset between the centroids is 2 mm, or less than 1 mm. In other applications, the magnetic centroid M_{c} is at least approximately coincident with the radiographic centroid R_{c} when the centroids are spaced apart by a distance not greater than half the length of the magnetic transponder 120 and/or the marker 100.

The imaging element 130 can be made from a material and configured appropriately to absorb a high fraction of incident photons of a radiation beam used for producing the radiographic image. For example, when the imaging radiation has high acceleration voltages in the megavoltage range, the imaging element 130 is made from, at least in part, high density materials with sufficient thickness and cross-sectional area to absorb enough of the photon fluence incident on the imaging element to be visible in the resulting radiograph. Many high energy beams used for therapy have acceleration voltages of 6 MV-25 MV, and these beams are often used to produce radiographic images in the 5 MV-10 MV range, or more specifically in the 6 MV-8 MV range. As such, the imaging element 130 can be made from a material that is sufficiently absorbent of incident photon fluence to be visible in an image produced using a beam with an acceleration voltage of 5 MV-10 MV, or more specifically an acceleration voltage of 6 MV-8 MV.

Several specific embodiments of imaging elements 130 can be made from gold, tungsten, platinum and/or other high density metals. In these embodiments the imaging element 130 can be composed of materials having a density of 19.25 g/cm3 (density of tungsten) and/or a density of approximately 21.4 g/cm3 (density of platinum). Many embodiments of the imaging element 130 accordingly have a density not less than 19 g/cm3. In other embodiments, however, the material(s) of the imaging element 130 can have a substantially lower density. For example, imaging elements with lower density materials are suitable for applications that use lower energy radiation to produce radiographic images. Moreover, the first and second contrast elements 132 and 134 can be composed of different materials such that the first contrast element 132 can be made from a first material and the second contrast element 134 can be made from a second material.

Referring to FIG. 12B, the marker 100 can further include a module 140 at an opposite end of the core 128 from the capacitor 126. In the embodiment of the marker 100 shown in FIG. 12B, the module 140 is configured to be symmetrical with respect to the capacitor 126 to enhance the symmetry of the radiographic image. As with the first and second contrast elements 132 and 134, the module 140 and the capacitor 126 are arranged such that the magnetic centroid of the marker is at least approximately coincident with the radiographic centroid of the marker 100. The module 140 can be another capacitor that is identical to the capacitor 126, or the module 140 can be an electrically inactive element. Suitable electrically inactive modules include ceramic blocks shaped like the capacitor 126 and located with respect to the coil 122, the core 128 and the imaging element 130 to be symmetrical with each other. In still other embodiments the module 140 can be a different type of electrically active element electrically coupled to the magnetic transponder 120.

One specific process of using the marker involves imaging the marker using a first modality and then tracking the target of the patient and/or the marker using a second modality. For example, the location of the marker relative to the target can be determined by imaging the marker and the target using radiation. The marker and/or the target can then be localized and tracked using the magnetic field generated by the marker in response to an excitation energy.

The marker 100 shown in FIGS. 12A-B is expected to provide an enhanced radiographic image compared to conventional magnetic markers for more accurately determining the relative position between the marker and the target of a patient. FIG. 12C, for example, illustrates a radiographic image 150 of the marker 100 and a target T of the patient. The first and second contrast elements 132 and 134 are expected to be more distinct in the radiographic image 150 because they can be composed of higher density materials than the components of the magnetic transponder 120. The first and second contrast elements 132 and 134 can accordingly appear as bulbous ends of a dumbbell shape in applications in which the components of the magnetic transponder 120 are visible in the image. In certain megavolt applications, the components of the magnetic transponder 120 may not appear at all on the radiographic image 150 such that the first and second contrast elements 132 and 134 will appear as distinct regions that are separate from each other. In either embodiment, the first and second contrast elements 132 and 134 provide a reference frame in which the radiographic centroid R_{c} of the marker 100 can be located in the image 150. Moreover, because the imaging element 130 is configured so that the radiographic centroid R_{c} is at least approximately coincident with the magnetic centroid M_{c}, the relative offset or position between the target T and the magnetic centroid M_{c} can be accurately determined using the marker 100. The embodiment of the marker 100 illustrated in FIGS. 12A-C, therefore, is expected to mitigate errors caused by incorrectly estimating the radiographic and magnetic centroids of markers in radiographic images.

FIG. 13A is an isometric view of a marker 200 with a cut-away portion to illustrate internal components, and FIG. 13B is a cross-sectional view of the marker 200 taken along line 13B-13B of FIG. 13A. The marker 200 is similar to the marker 100 shown above in FIG. 12A, and thus like reference numbers refer to like components. The marker 200 differs from the marker 100 in that the marker 200 includes an imaging element 230 defined by a single contrast element. The imaging element 230 is generally configured relative to the magnetic transponder 120 so that the radiographic centroid of the marker 200 is at least approximately coincident with the magnetic centroid of the magnetic transponder 120. The imaging element 230, more specifically, is a ring extending around the coil 122 at a medial region of the magnetic transponder 120. The imaging element 230 can be composed of the same materials described above with respect to the imaging element 130 in FIGS. 12A-B. The imaging element 230 can have an inner diameter that is approximately equal to the outer diameter of the coil 122, and an outer diameter within the casing 110. As shown in FIG. 13B, however, a spacer 231 can be between the inner diameter of the imaging element 230 and the outer diameter of the coil 122.

The marker 200 is expected to operate in a manner similar to the marker 100 described above. The marker 200, however, does not have two separate contrast elements that provide two distinct, separate points in a radiographic image. The imaging element 230 is still highly useful in that it identifies the radiographic centroid of the marker 200 in a radiographic image, and it can be configured so that the radiographic centroid of the marker 200 is at least approximately coincident with the magnetic centroid of the magnetic transponder 120.

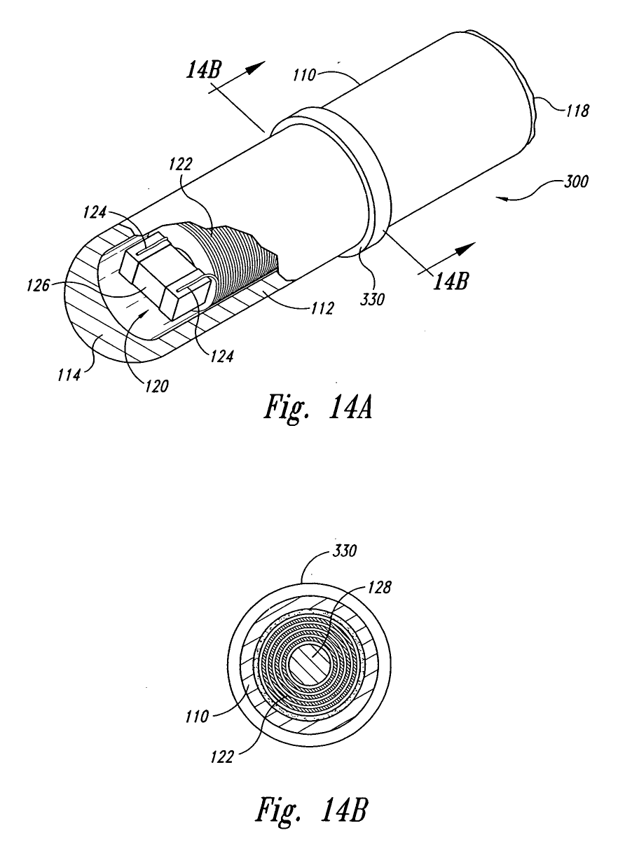

FIG. 14A is an isometric view of a marker 300 having a cut-away portion, and FIG. 14B is a cross-sectional view of the marker 300 taken along line 14B-14B of FIG. 14A. The marker 300 is substantially similar to the marker 200 shown in FIGS. 13A-B, and thus like reference numbers refer to like components in FIGS. 12A-14B. The imaging element 330 can be a high density ring configured relative to the magnetic transponder 120 so that the radiographic centroid of the marker 300 is at least approximately coincident with the magnetic centroid of the magnetic transponder 120. The marker 300, more specifically, includes an imaging element 330 around the casing 110. The marker 300 is expected to operate in much the same manner as the marker 200 shown in FIGS. 13A-B.

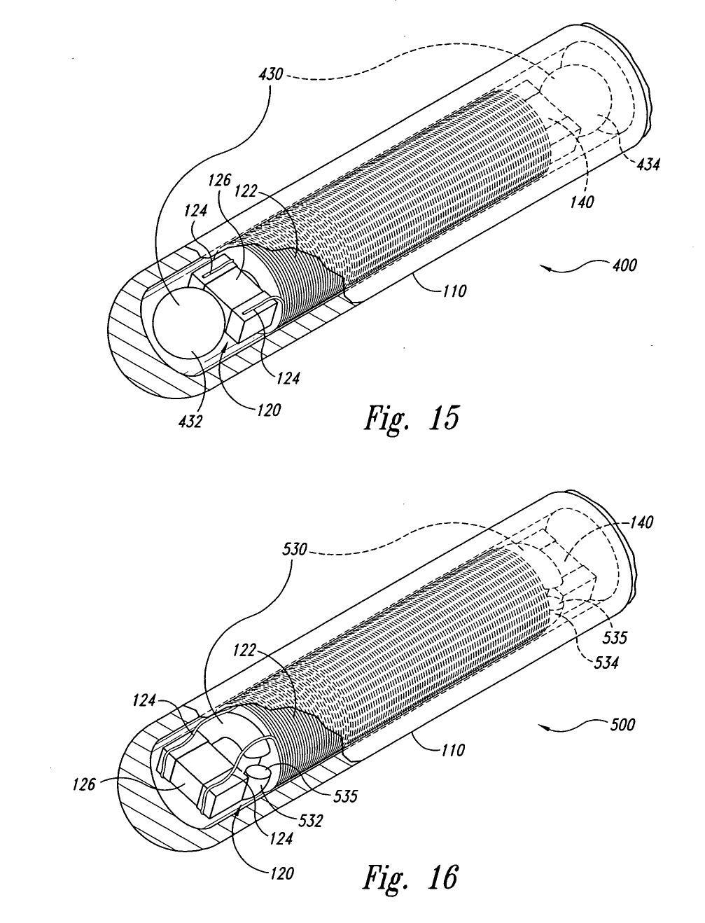

FIG. 15 is an isometric view with a cut-away portion illustrating a marker 400 in accordance with another embodiment of the invention. The marker 400 is similar to the marker 100 shown in FIGS. 12A-C, and thus like reference numbers refer to like components in these Figures. The marker 400 has an imaging element 430 including a first contrast element 432 at one end of the magnetic transponder 120 and a second contrast element 434 at another end of the magnetic transponder 120. The first and second contrast elements 432 and 434 are spheres composed of suitable high density materials. The contrast elements 432 and 434, for example, can be composed of gold, tungsten, platinum or other suitable high-density materials for use in radiographic imaging. The marker 400 is expected to operate in a manner similar to the marker 100, as described above.

FIG. 16 is an isometric view with a cut-away portion of a marker 500 in accordance with yet another embodiment of the invention. The marker 500 is substantially similar to the markers 100 and 400 shown in FIGS. 12A and 15, and thus like reference numbers refer to like components in these Figures. The marker 500 includes an imaging element 530 including a first contrast element 532 and a second contrast element 534. The first and second contrast elements 532 and 534 can be positioned proximate to opposing ends of the magnetic transponder 120. The first and second contrast elements 532 and 534 can be discontinuous rings having a gap 535 to mitigate eddy currents. The contrast elements 532 and 534 can be composed of the same materials as described above with respect to the contrast elements of other imaging elements in accordance with other embodiments of the invention.

Additional embodiments of markers in accordance with the invention can include imaging elements incorporated into or otherwise integrated with the casing 110, the core 128 (FIG. 12B) of the magnetic transponder 120, and/or the adhesive 129 (FIG. 12B) in the casing. For example, particles of a high density material can be mixed with ferrite and extruded to form the core 128. Alternative embodiments can mix particles of a high density material with glass or another material to form the casing 110, or coat the casing 110 with a high-density material. In still other embodiments, a high density material can be mixed with the adhesive 129 and injected into the casing 110. Any of these embodiments can incorporate the high density material into a combination of the casing 110, the core 128 and/or the adhesive 129. Suitable high density materials can include tungsten, gold and/or platinum as described above.

The markers described above with reference to FIGS. 12A-16 can be used for the markers 40 in the localization system 10 (FIGS. 1-7). The localization system 10 can have several markers with the same type of imaging elements, or markers with different imaging elements can be used with the same instrument. Several additional details of these markers and other embodiments of markers are described in U.S. application Ser. Nos. 10/334,698 and 10/746,888, which are incorporated herein by reference. For example, the markers may not have any imaging elements for applications with lower energy radiation, or the markers may have reduced volumes of ferrite and metals to mitigate issues with MRI imaging as set forth in U.S. application Ser. No. 10/334,698.

2. Localization Systems

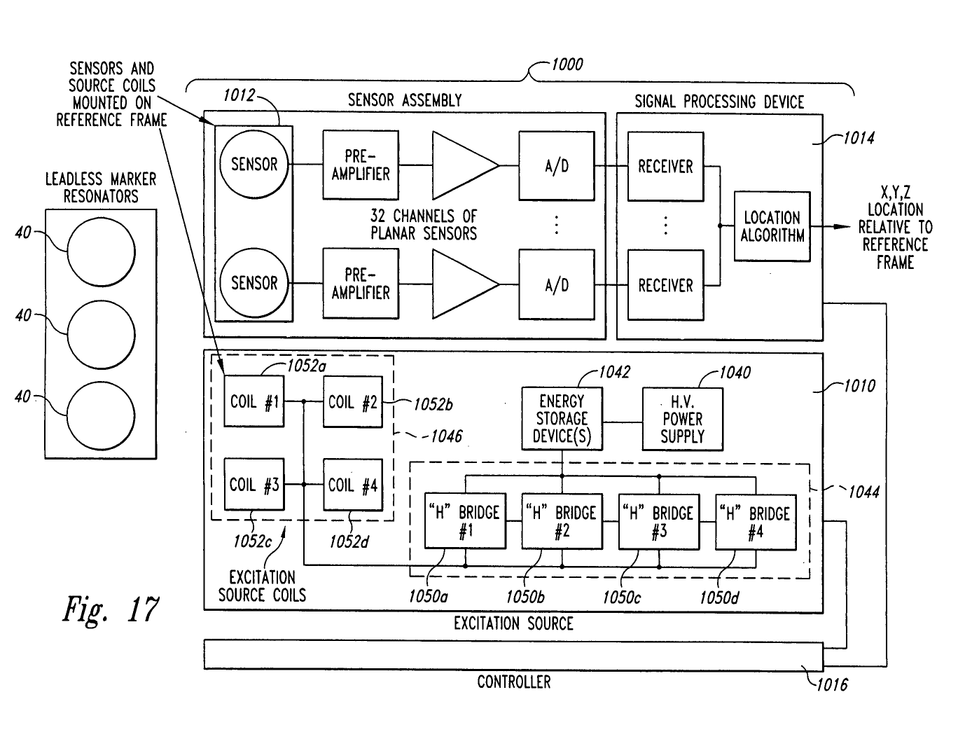

FIG. 17 is a schematic block diagram of a localization system 1000 for determining the absolute location of the markers 40 (shown schematically) relative to a reference frame. The localization system 1000 includes an excitation source 1010, a sensor assembly 1012, a signal processor 1014 operatively coupled to the sensor assembly 1012, and a controller 1016 operatively coupled to the excitation source 1010 and the signal processor 1014. The excitation source 1010 is one embodiment of the excitation source 60 described above with reference to FIG. 3; the sensor assembly 1012 is one embodiment of the sensor assembly 70 described above with reference to FIG. 3; and the controller 1016 is one embodiment of the controller 80 described above with reference to FIG. 3.

The excitation source 1010 is adjustable to generate a magnetic field having a waveform with energy at selected frequencies to match the resonant frequencies of the markers 40. The magnetic field generated by the excitation source 1010 energizes the markers at their respective frequencies. After the markers 40 have been energized, the excitation source 1010 is momentarily switched to an “off” position so that the pulsed magnetic excitation field is terminated while the markers wirelessly transmit the location signals. This allows the sensor assembly 1012 to sense the location signals from the markers 40 without measurable interference from the significantly more powerful magnetic field from the excitation source 1010. The excitation source 1010 accordingly allows the sensor assembly 1012 to measure the location signals from the markers 40 at a sufficient signal-to-noise ratio so that the signal processor 1014 or the controller 1016 can accurately calculate the absolute location of the markers 40 relative to a reference frame.

a. Excitation Sources

Referring still to FIG. 17, the excitation source 1010 includes a high voltage power supply 1040, an energy storage device 1042 coupled to the power supply 1040, and a switching network 1044 coupled to the energy storage device 1042. The excitation source 1010 also includes a coil assembly 1046 coupled to the switching network 1044. In one embodiment, the power supply 1040 is a 500 volt power supply, although other power supplies with higher or lower voltages can be used. The energy storage device 1042 in one embodiment is a high voltage capacitor that can be charged and maintained at a relatively constant charge by the power supply 1040. The energy storage device 1042 alternately provides energy to and receives energy from the coils in the coil assembly 1046.

The energy storage device 1042 is capable of storing adequate energy to reduce voltage drop in the energy storage device while having a low series resistance to reduce power losses. The energy storage device 1042 also has a low series inductance to more effectively drive the coil assembly 1046. Suitable capacitors for the energy storage device 1042 include aluminum electrolytic capacitors used in flash energy applications. Alternative energy storage devices can also include NiCd and lead acid batteries, as well as alternative capacitor types, such as tantalum, film, or the like.

The switching network 1044 includes individual H-bridge switches 1050 (identified individually by reference numbers 1050a-d), and the coil assembly 1046 includes individual source coils 1052 (identified individually by reference numbers 1052a-d). Each H-bridge switch 1050 controls the energy flow between the energy storage device 1042 and one of the source coils 1052. For example, H-bridge switch #1 1050a independently controls the flow of the energy to/from source coil #1 1052a, H-bridge switch #2 1050b independently controls the flow of the energy to/from source coil #2 1052b, H-bridge switch #3 1050c independently controls the flow of the energy to/from source coil #3 1052c, and H-bridge switch #4 1050d independently controls the flow of the energy to/from source coil #4 1052d. The switching network 1044 accordingly controls the phase of the magnetic field generated by each of the source coils 1052a-d independently. The H-bridges 1050 can be configured so that the electrical signals for all the source coils 1052 are in phase, or the H-bridge switches 1050 can be configured so that one or more of the source coils 1052 are 180° out of phase. Furthermore, the H-bridge switches 1050 can be configured so that the electrical signals for one or more of the source coils 1052 are between 0 and 180° out of phase to simultaneously provide magnetic fields with different phases.

The source coils 1052 can be arranged in a coplanar array that is fixed relative to the reference frame. Each source coil 1052 can be a square, planar winding arranged to form a flat, substantially rectilinear coil. The source coils 1052 can have other shapes and other configurations in different embodiments. In one embodiment, the source coils 1052 are individual conductive lines formed in a stratum of a printed circuit board, or windings of a wire in a foam frame. Alternatively, the source coils 1052 can be formed in different substrates or arranged so that two or more of the source coils are not planar with each other. Additionally, alternate embodiments of the invention may have fewer or more source coils than illustrated in FIG. 17.

The selected magnetic fields from the source coils 1052 combine to form an adjustable excitation field that can have different three-dimensional shapes to excite the markers 40 at any spatial orientation within an excitation volume. When the planar array of the source coils 1052 is generally horizontal, the excitation volume is positioned above an area approximately corresponding to the central region of the coil assembly 1046. The excitation volume is the three-dimensional space adjacent to the coil assembly 1046 in which the strength of the magnetic field is sufficient to adequately energize the markers 40.

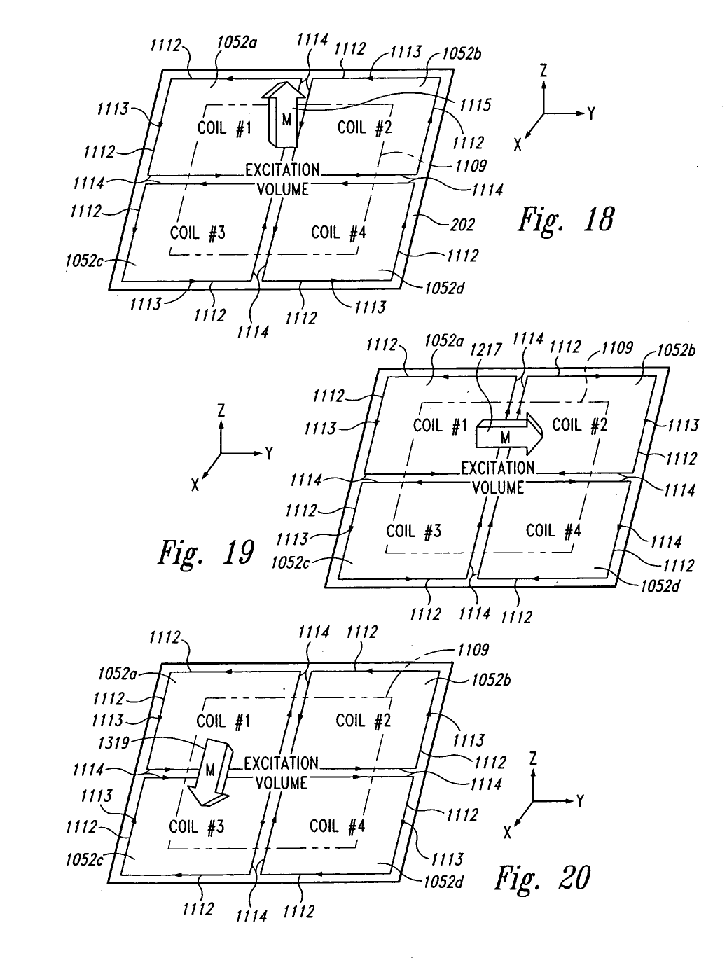

FIGS. 18-20 are schematic views of a planar array of the source coils 1052 with the alternating electrical signals provided to the source coils in different combinations of phases to generate excitation fields about different axes relative to the illustrated XYZ coordinate system. Each source coil 1052 has two outer sides 1112 and two inner sides 1114. Each inner side 1114 of one source coil 1052 is immediately adjacent to an inner side 1114 of another source coil 1052, but the outer sides 1112 of all the source coils 1052 are not adjacent to any other source coil 1052.

In the embodiment of FIG. 18, all the source coils 1052a-d simultaneously receive an alternating electrical signals in the same phase. As a result, the electrical current flows in the same direction through all the source coils 1052a-d such that a direction 1113 of the current flowing along the inner sides 1114 of one source coil (e.g., source coil 1052a) is opposite to the direction 1113 of the current flowing along the inner sides 1114 of the two adjacent source coils (e.g., source coils 1052c and 1052d). The magnetic fields generated along the inner sides 1114 accordingly cancel each other out so that the magnetic field is effectively generated from the current flowing along the outer sides 1112 of the source coils. The resulting excitation field formed by the combination of the magnetic fields from the source coils 1052a-d shown in FIG. 18 has a magnetic moment 1115 generally in the Z direction within an excitation volume 1109. This excitation field energizes markers parallel to the Z-axis or markers positioned with an angular component along the Z-axis (i.e., not orthogonal to the Z-axis).

FIG. 19 is a schematic view of the source coils 1052a-d with the alternating electrical signals provided in a second combination of phases to generate a second excitation field with a different spatial orientation. In this embodiment, source coils 1052a and 1052c are in phase with each other, and source coils 1052b and 1052d are in phase with each other. However, source coils 1052a and 1052c are 180 degrees out of phase with source coils 1052b and 1052d. The magnetic fields from the source coils 1052a-d combine to generate an excitation field having a magnetic moment 1217 generally in the Y direction within the excitation volume 1109. Accordingly, this excitation field energizes markers parallel to the Y-axis or markers positioned with an angular component along the Y-axis.

FIG. 20 is a schematic view of the source coils 1052a-d with the alternating electrical signals provided in a third combination of phases to generate a third excitation field with a different spatial orientation. In this embodiment, source coils 1052a and 1052b are in phase with each other, and source coils 1052c and 1052d are in phase with each other. However, source coils 1052a and 1052b are 180 degrees out of phase with source coils 1052c and 1052d. The magnetic fields from the source coils 1052a-d combine to generate an excitation field having a magnetic moment 1319 in the excitation volume 1109 generally in the direction of the X-axis. Accordingly, this excitation field energizes markers parallel to the X-axis or markers positioned with an angular component along the X-axis.

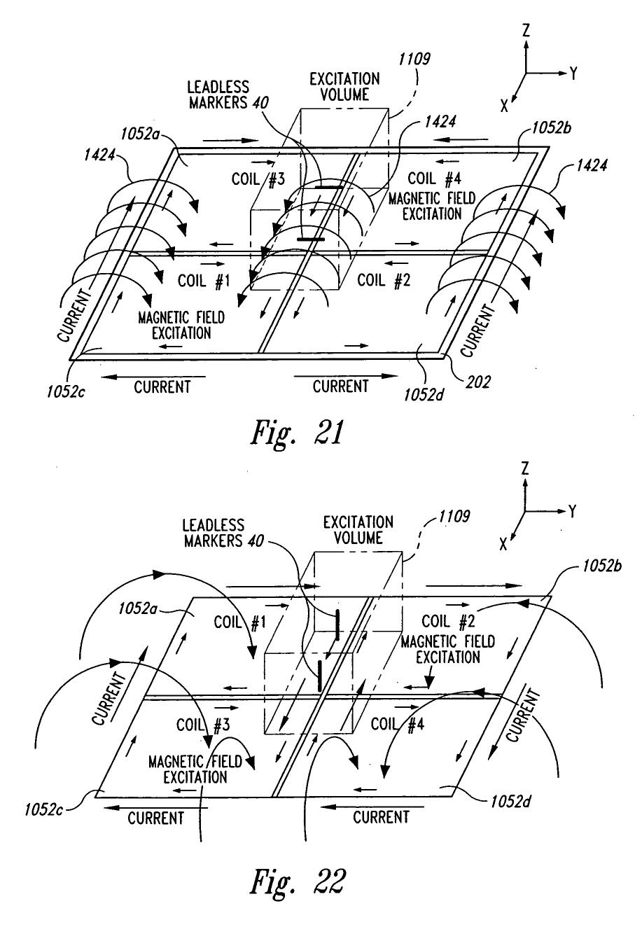

FIG. 21 is a schematic view of the source coils 1052a-d illustrating the current flow to generate an excitation field 1424 for energizing markers 40 with longitudinal axes parallel to the Y-axis. The switching network 1044 (FIG. 17) is configured so that the phases of the alternating electrical signals provided to the source coils 1052a-d are similar to the configuration of FIG. 18. This generates the excitation field 1424 with a magnetic moment in the Y direction to energize the markers 40.

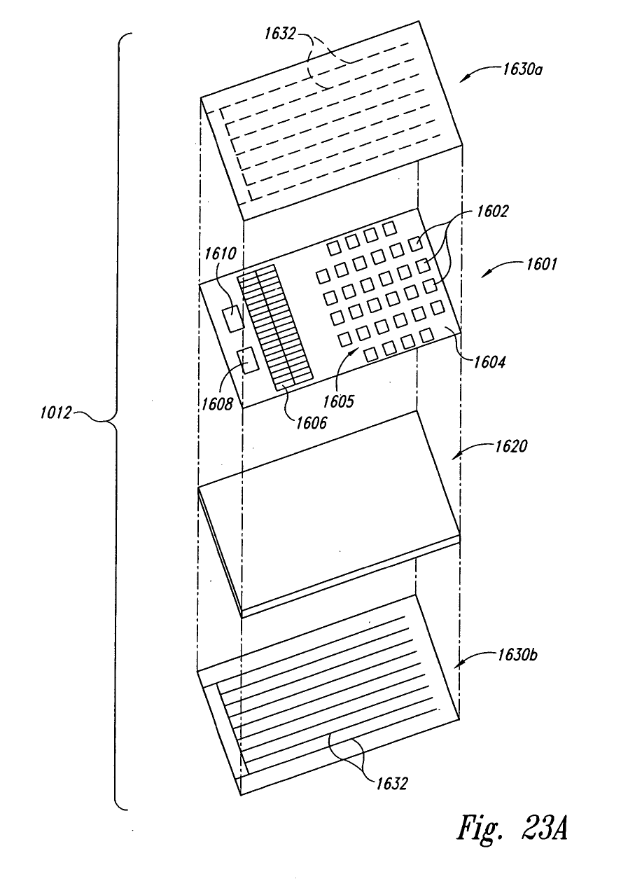

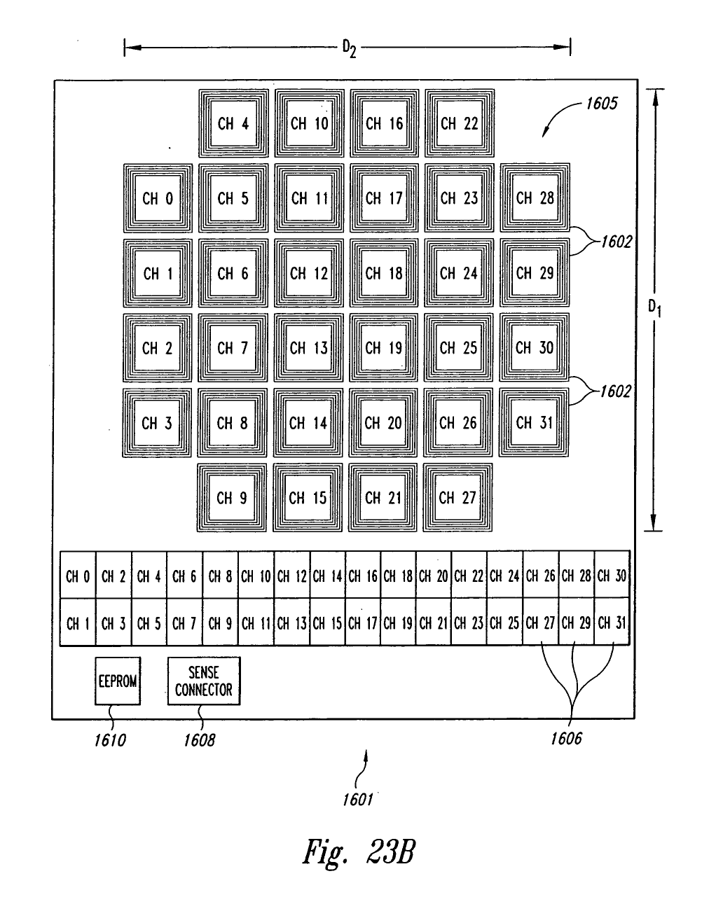

FIG. 22 further illustrates the ability to spatially adjust the excitation field in a manner that energizes any of the markers 40 at different spatial orientations. In this embodiment, the switching network 1044 (FIG. 17) is configured so that the phases of the alternating electrical signals provided to the source coils 1052a-d are similar to the configuration shown in FIG. 18. This produces an excitation field with a magnetic moment in the Z direction that energizes markers 40 with longitudinal axes parallel to the Z-axis.

The spatial configuration of the excitation field in the excitation volume 1109 can be quickly adjusted by manipulating the switching network to change the phases of the electrical signals provided to the source coils 1052a-d. As a result, the overall magnetic excitation field can be changed to be oriented in either the X, Y or Z directions within the excitation volume 1109. This adjustment of the spatial orientation of the excitation field reduces or eliminates blind spots in the excitation volume 1109. Therefore, the markers 40 within the excitation volume 1109 can be energized by the source coils 1052a-d regardless of the spatial orientations of the leadless markers.

In one embodiment, the excitation source 1010 is coupled to the sensor assembly 1012 so that the switching network 1044 (FIG. 17) adjusts orientation of the pulsed generation of the excitation field along the X, Y, and Z axes depending upon the strength of the signal received by the sensor assembly. If the location signal from a marker 40 is insufficient, the switching network 1044 can automatically change the spatial orientation of the excitation field during a subsequent pulsing of the source coils 1052a-d to generate an excitation field with a moment in the direction of a different axis or between axes. The switching network 1044 can be manipulated until the sensor assembly 1012 receives a sufficient location signal from the marker.

The excitation source 1010 illustrated in FIG. 17 alternately energizes the source coils 1052a-d during an excitation phase to power the markers 40, and then actively de-energizes the source coils 1052a-d during a sensing phase in which the sensor assembly 1012 senses the decaying location signals wirelessly transmitted by the markers 40. To actively energize and de-energize the source coils 1052a-d, the switching network 1044 is configured to alternatively transfer stored energy from the energy storage device 1042 to the source coils 1052a-d, and to then re-transfer energy from the source coils 1052a-d back to the energy storage device 1042. The switching network 1044 alternates between first and second “on” positions so that the voltage across the source coils 1052 alternates between positive and negative polarities. For example, when the switching network 1044 is switched to the first “on” position, the energy in the energy storage device 1042 flows to the source coils 1052a-d. When the switching network 1044 is switched to the second “on” position, the polarity is reversed such that the energy in the source coils 1052a-d is actively drawn from the source coils 1052a-d and directed back to the energy storage device 1042. As a result, the energy in the source coils 1052a-d is quickly transferred back to the energy storage device 1042 to abruptly terminate the excitation field transmitted from the source coils 1052a-d and to conserve power consumed by the energy storage device 1042. This removes the excitation energy from the environment so that the sensor assembly 1012 can sense the location signals from the markers 40 without interference from the significantly larger excitation energy from the excitation source 1010. Several additional details of the excitation source 1010 and alternate embodiments are disclosed in U.S. patent application Ser. No. 10/213,980 filed on Aug. 7, 2002, which is incorporated by reference herein in its entirety.

b. Sensor Assemblies

FIG. 23A is an exploded isometric view showing several components of the sensor assembly 1012 for use in the localization system 1000 (FIG. 17). The sensor assembly 1012 includes a sensing unit 1601 having a plurality of coils 1602 formed on or carried by a panel 1604. The coils 1602 can be field sensors or magnetic flux sensors arranged in a sensor array 1605.

The panel 1604 may be a substantially non-conductive material, such as a sheet of KAPTON® produced by DuPont. KAPTON® is particularly useful when an extremely stable, tough, and thin film is required (such as to avoid radiation beam contamination), but the panel 1604 may be made from other materials and have other configurations. For example, FR4 (epoxy-glass substrates), GETEK or other Teflon-based substrates, and other commercially available materials can be used for the panel 1604. Additionally, although the panel 1604 may be a flat, highly planar structure, in other embodiments, the panel may be curved along at least one axis. In either embodiment, the field sensors (e.g., coils) are arranged in a locally planar array in which the plane of one field sensor is at least substantially coplanar with the planes of adjacent field sensors. For example, the angle between the plane defined by one coil relative to the planes defined by adjacent coils can be from approximately 0° to 10°, and more generally is less than 5°. In some circumstances, however, one or more of the coils may be at an angle greater than 10° relative to other coils in the array.

The sensor assembly 1012 shown in FIG. 23A can optionally include a core 1620 laminated to the panel 1604. The core 1620 can be a support member made from a rigid material, or the core 1620 can be a low density foam, such as a closed-cell Rohacell foam. The core 1620 is preferably a stable layer that has a low coefficient of thermal expansion so that the shape of the sensor assembly 1012 and the relative orientation between the coils 1602 remain within a defined range over an operating temperature range.

The sensor assembly 1012 can further include a first exterior cover 1630a on one side of the sensing subsystem and a second exterior cover 1630b on an opposing side. The first and second exterior covers 1630a-b can be thin, thermally stable layers, such as Kevlar or Thermount films. Each of the first and second exterior covers 1630a-b can include electric shielding 1632 to block undesirable external electric fields from reaching the coils 1602. The electric shielding 1632 can be a plurality of parallel legs of gold-plated, copper strips to define a comb-shaped shield in a configuration commonly called a Faraday shield. It will be appreciated that the shielding can be formed from other materials that are suitable for shielding. The electric shielding can be formed on the first and second exterior covers using printed circuit board manufacturing technology or other techniques.

The panel 1604 with the coils 1602 is laminated to the core 1620 using a pressure sensitive adhesive or another type of adhesive. The first and second exterior covers 1630a-b are similarly laminated to the assembly of the panel 1604 and the core 1620. The laminated assembly forms a rigid structure that fixedly retains the arrangement of the coils 1602 in a defined configuration over a large operating temperature range. As such, the sensor assembly 1012 does not substantially deflect across its surface during operation. The sensor assembly 1012, for example, can retain the array of coils 1602 in the fixed position with a deflection of no greater than ±0.5 mm, and in some cases no more than ±0.3 mm. The stiffness of the sensing subsystem provides very accurate and repeatable monitoring of the precise location of leadless markers in real time.

In still another embodiment, the sensor assembly 1012 can further include a plurality of source coils that are a component of the excitation source 1010. One suitable array combining the sensor assembly 1012 with source coils is disclosed in U.S. patent application Ser. No. 10/334,700, entitled PANEL-TYPE SENSOR/SOURCE ARRAY ASSEMBLY, filed on Dec. 30, 2002, which is herein incorporated by reference.

FIG. 23B further illustrates an embodiment of the sensing unit 1601. In this embodiment, the sensing unit 1601 includes 32 sensor coils 1602; each coil 1602 is associated with a separate channel 1606 (shown individually as channels “Ch 0” through “Ch 31”). The overall dimension of the panel 1604 can be approximately 40 cm by 54 cm, but the array 1605 has a first dimension D1 of approximately 40 cm and a second dimension D2 of approximately 40 cm. The array 1605 can have other sizes or other configurations (e.g., circular) in alternative embodiments. Additionally, the array 1605 can have more or fewer coils, such as 8-64 coils; the number of coils may moreover be a power of 2.

The coils 1602 may be conductive traces or depositions of copper or another suitably conductive metal formed on the panel 1604. Each coil 1602 has a trace with a width of approximately 0.15 mm and a spacing between adjacent turns within each coil of approximately 0.13 mm. The coils 1602 can have approximately 15 to 90 turns, and in specific applications each coil has approximately 40 turns. Coils with less than 15 turns may not be sensitive enough for some applications, and coils with more than 90 turns may lead to excessive voltage from the source signal during excitation and excessive settling times resulting from the coil's lower self-resonant frequency. In other applications, however, the coils 1602 can have less than 15 turns or more than 90 turns.

As shown in FIG. 23B, the coils 1602 are arranged as square spirals, although other configurations may be employed, such as arrays of circles, interlocking hexagons, triangles, etc. Such square spirals utilize a large percentage of the surface area to improve the signal to noise ratio. Square coils also simplify design layout and modeling of the array compared to circular coils; for example, circular coils could waste surface area for linking magnetic flux from the markers 40. The coils 1602 have an inner dimension of approximately 40 mm, and an outer dimension of approximately 62 mm, although other dimensions are possible depending upon applications. Sensitivity may be improved with an inner dimension as close to an outer dimension as possible given manufacturing tolerances. In several embodiments, the coils 1602 are identical to each other or at least configured substantially similarly.

The pitch of the coils 1602 in the array 1605 is a function of, at least in part, the minimum distance between the marker and the coil array. In one embodiment, the coils are arranged at a pitch of approximately 67 mm. This specific arrangement is particularly suitable when the wireless markers 40 are positioned approximately 7-27 cm from the sensor assembly 1012. If the wireless markers are closer than 7 cm, then the sensing subsystem may include sensor coils arranged at a smaller pitch. In general, a smaller pitch is desirable when wireless markers are to be sensed at a relatively short distance from the array of coils. The pitch of the coils 1602, for example, is approximately 50%-200% of the minimum distance between the marker and the array.

In general, the size and configuration of the array 1605 and the coils 1602 in the array depend on the frequency range in which they are to operate, the distance from the markers 40 to the array, the signal strength of the markers, and several other factors. Those skilled in the relevant art will readily recognize that other dimensions and configurations may be employed depending, at least in part, on a desired frequency range and distance from the markers to the coils.

The array 1605 is sized to provide a large aperture to measure the magnetic field emitted by the markers. It can be particularly challenging to accurately measure the signal emitted by an implantable marker that wirelessly transmits a marker signal in response to a wirelessly transmitted energy source because the marker signal is much smaller than the source signal and other magnetic fields in a room (e.g., magnetic fields from CRTs, etc.). The size of the array 1605 can be selected to preferentially measure the near field of the marker while mitigating interference from far field sources. In one embodiment, the array 1605 is sized to have a maximum dimension D1 or D2 across the surface of the area occupied by the coils that is approximately 100% to 300% of a predetermined maximum sensing distance that the markers are to be spaced from the plane of the coils. Thus, the size of the array 1605 is determined by identifying the distance that the marker is to be spaced apart from the array to accurately measure the marker signal, and then arrange the coils so that the maximum dimension of the array is approximately 100% to 300% of that distance. The maximum dimension of the array 1605, for example, can be approximately 200% of the sensing distance at which a marker is to be placed from the array 1605. In one specific embodiment, the marker 40 has a sensing distance of 20 cm and the maximum dimension of the array of coils 1602 is between 20 cm and 60 cm, and more specifically 40 cm.

A coil array with a maximum dimension as set forth above is particularly useful because it inherently provides a filter that mitigates interference from far field sources. As such, one aspect of several embodiments of the invention is to size the array based upon the signal from the marker so that the array preferentially measures near field sources (i.e., the field generated by the marker) and filters interference from far field sources.

The coils 1602 are electromagnetic field sensors that receive magnetic flux produced by the wireless markers 40 and in turn produce a current signal representing or proportional to an amount or magnitude of a component of the magnetic field through an inner portion or area of each coil. The field component is also perpendicular to the plane of each coil 1602. Each coil represents a separate channel, and thus each coil outputs signals to one of 32 output ports 1606. A preamplifier, described below, may be provided at each output port 1606. Placing preamplifiers (or impedance buffers) close to the coils minimizes capacitive loading on the coils, as described herein. Although not shown, the sensing unit 1601 also includes conductive traces or conductive paths routing signals from each coil 1602 to its corresponding output port 1606 to thereby define a separate channel. The ports in turn are coupled to a connector 1608 formed on the panel 1604 to which an appropriately configured plug and associated cable may be attached.

The sensing unit 1601 may also include an onboard memory or other circuitry, such as shown by electrically erasable programmable read-only memory (EEPROM) 1610. The EEPROM 1610 may store manufacturing information such as a serial number, revision number, date of manufacture, and the like. The EEPROM 1610 may also store per-channel calibration data, as well as a record of run-time. The run-time will give an indication of the total radiation dose to which the array has been exposed, which can alert the system when a replacement sensing subsystem is required.

Although shown in one plane only, additional coils or electromagnetic field sensors may be arranged perpendicular to the panel 1604 to help determine a three-dimensional location of the wireless markers 40. Adding coils or sensors in other dimensions could increase the total energy received from the wireless markers 40, but the complexity of such an array would increase disproportionately. The inventors have found that three-dimensional coordinates of the wireless markers 40 may be found using the planar array shown in FIG. 23A-B.

Implementing the sensor assembly 1012 may involve several considerations. First, the coils 1602 may not be presented with an ideal open circuit. Instead, they may well be loaded by parasitic capacitance due largely to traces or conductive paths connecting the coils 1602 to the preamplifiers, as well as a damping network (described below) and an input impedance of the preamplifiers (although a low input impedance is preferred). These combined loads result in current flow when the coils 1602 link with a changing magnetic flux. Any one coil 1602, then, links magnetic flux not only from the wireless marker 40, but also from all the other coils as well. These current flows should be accounted for in downstream signal processing.

A second consideration is the capacitive loading on the coils 1602. In general, it is desirable to minimize the capacitive loading on the coils 1602. Capacitive loading forms a resonant circuit with the coils themselves, which leads to excessive voltage overshoot when the excitation source 1010 is energized. Such a voltage overshoot should be limited or attenuated with a damping or “snubbing” network across the coils 1602. A greater capacitive loading requires a lower impedance damping network, which can result in substantial power dissipation and heating in the damping network.

Another consideration is to employ preamplifiers that are low noise. The preamplification can also be radiation tolerant because one application for the sensor assembly 1012 is with radiation therapy systems that use linear accelerators (LINAC). As a result, PNP bipolar transistors and discrete elements may be preferred. Further, a DC coupled circuit may be preferred if good settling times cannot be achieved with an AC circuit or output, particularly if analog to digital converters are unable to handle wide swings in an AC output signal.

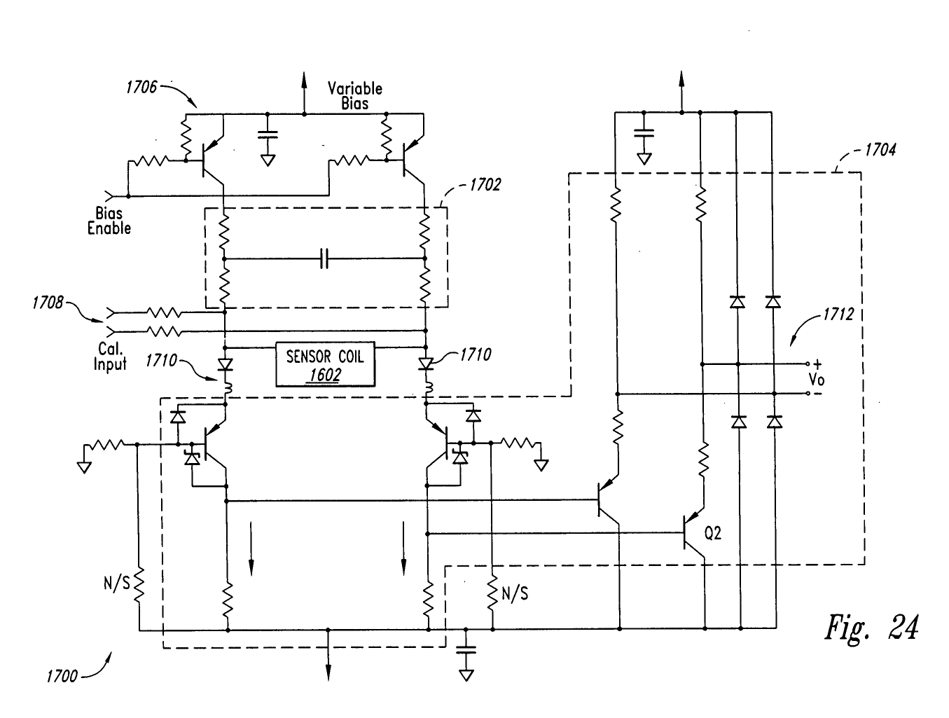

FIG. 24, for example, illustrates an embodiment of a snubbing network 1702 having a differential amplifier 1704. The snubbing network 1702 includes two pairs of series coupled resistors and a capacitor bridging therebetween. A biasing circuit 1706 allows for adjustment of the differential amplifier, while a calibration input 1708 allows both input legs of the differential amplifier to be balanced. The coil 1602 is coupled to an input of the differential amplifier 1704, followed by a pair of high voltage protection diodes 1710. DC offset may be adjusted by a pair of resistors coupled to bases of the input transistors for the differential amplifier 1704 (shown as having a zero value). Additional protection circuitry is provided, such as ESD protection diodes 1712 at the output, as well as filtering capacitors (shown as having a 10 nF value).

c. Signal Processors and Controllers

The signal processor 1014 and the controller 1016 illustrated in FIG. 17 receive the signals from the sensor assembly 1012 and calculate the absolute positions of the markers 40 within the reference frame. Suitable signal processing systems and algorithms are set forth in U.S. application Ser. Nos. 10/679,801; 10/749,478; 10/750,456; 10/750,164; 10/750,165; 10/749,860; and 10/750,453, all of which are incorporated herein by reference.

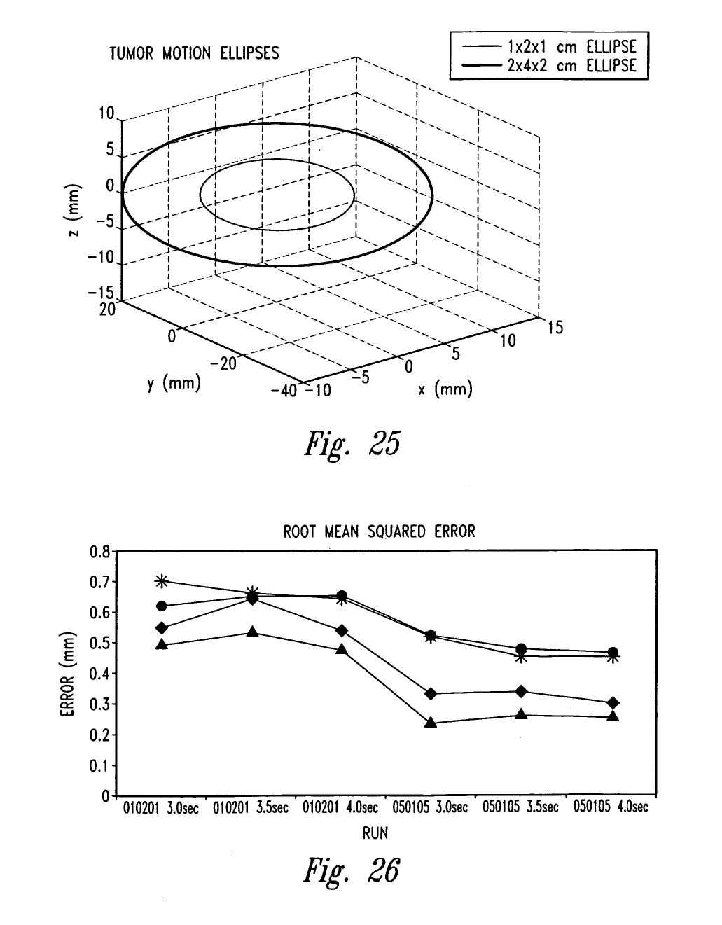

An experimental phantom based study was conducted to determine effectiveness of this system for real-time tracking. In this experiment, a custom 4D stage was constructed to allow arbitrary motion in three axes for speeds up to 10 cm/sec in each dimension, with accuracy to 0.3 mm. Position accuracy was measured by a 3D digitizing arm attached to the stage system. As shown in FIG. 25, two ellipses were created with peak to peak motion of 2 cm, 4 cm and 2 cm; and 1 cm by 2 cm and 1 cm in the x, y and z direction respectively. Three periods were used to correspond to 15, 17 and 20 breaths per minute. A single transponder was used with an integration time of 33 ms, 67 ms and 100 ms and two transponders were used with integration times of 67 ms and 100 ms. The transponders were placed in a custom phantom mounted to the 4D stage. The experiment was performed with the isocenter placed 14 cm from the AC magnetic array to simulate the position of an average lung cancer patient. The 4D stage ran each trajectory while the real tracking system measured the transponder positions. Measured position was compared against the phantom position. The effects of ellipse size, speed, transponder number and integration time were characterized.

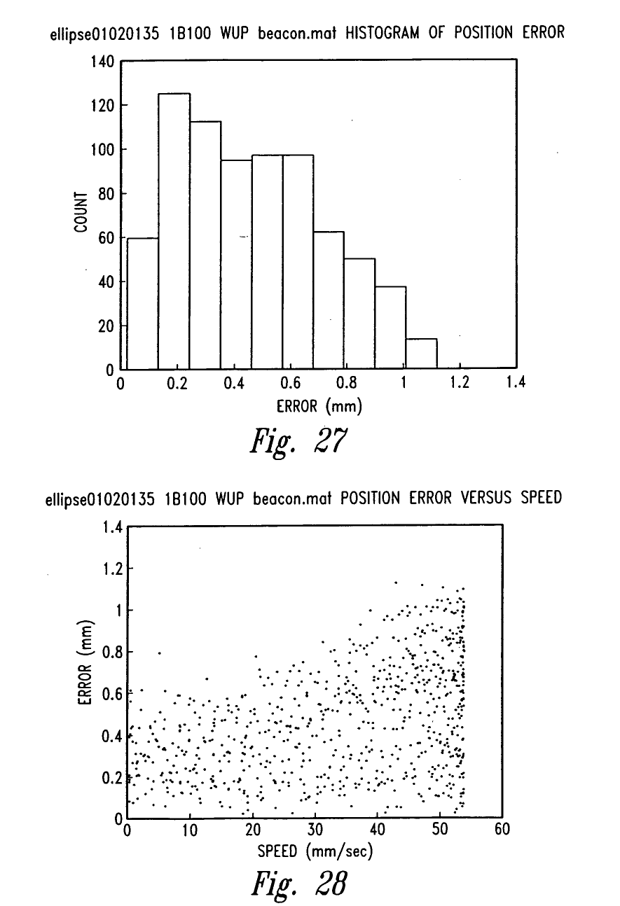

As shown in FIG. 26, the root mean square (RMS) error was less than 1 mm for each ellipse, period and transponder integration time. The system was able to track points throughout the path of the ellipse, for example, in a trajectory of a large ellipse moving at 17 breaths per minute. FIG. 27 is a histogram of localization errors illustrating that the range of error was low for each point measured. As shown in FIG. 28, the RMS error was higher in areas of increased velocity in most trajectories. With respect to this experiment, a single transponder system performed slightly better than dual transponder systems, with the best system being a single transponder with a 67 ms integration time.

The above description of illustrated embodiments, including what is described in the Abstract, is not intended to be exhaustive or to limit the invention to the precise forms disclosed. Although specific embodiments of and examples are described herein for illustrative purposes, various equivalent modifications can be made without departing from the spirit and scope of the invention, as will be recognized by those skilled in the relevant art. The teachings provided herein of the invention can be applied to target locating and tracking systems, not necessarily the exemplary system generally described above.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet, are incorporated herein by reference, in their entirety. Aspects of the invention can be modified, if necessary, to employ systems, devices and concepts of the various patents, applications and publications to provide yet further embodiments of the invention.

These and other changes can be made to the invention in light of the above-detailed description. In general, in the following claims, the terms used should not be construed to limit the invention to the specific embodiments disclosed in the specification and the claims, but should be construed to include all target locating and monitoring systems that operate in accordance with the claims to provide apparatus and methods for locating, monitoring, and/or tracking the position of a selected target within a body. Accordingly, the invention is not limited, except as by the appended claims.

I claim:

1. An apparatus for facilitating radiation treatment of a target in a patient, comprising:
a tube configured to receive a radiation source, the tube having a first end configured to be inserted into a patient and a second end;
an expandable member at the first end of the tube configured to contain the radiation source;
a marker associated with the expandable member such that the marker moves with the expandable member from a contracted orientation to an expanded orientation.

2. The apparatus of claim 1 wherein the expandable member comprises a balloon.

3. The apparatus of claim 1 wherein the expandable member comprises a flexible bladder.

4. The apparatus of claim 1 wherein the marker comprises a wireless transponder configured to wirelessly transmit a location signal in response to a wirelessly transmitted excitation energy.

5. The apparatus of claim 1 wherein the marker comprises a casing and a magnetic transponder in the casing, and wherein the magnetic transponder comprises a coil and a capacitor coupled to the coil.

6. The apparatus of claim 1 wherein the expandable member comprises a balloon, and wherein the apparatus further comprises a plurality of markers attached to the balloon.

7. The apparatus of claim 6 wherein the markers comprise wireless transponders configured to wirelessly transmit location signals in response to wirelessly transmitted excitation energy.

8. The apparatus of claim 6 wherein the markers comprise a first magnetic transponder having a first resonant frequency and a second magnetic transponder having a second resonant frequency different than the first resonant frequency.

9. The apparatus of claim 6 wherein the markers comprise radiopaque elements.

10. The apparatus of claim 1 wherein the apparatus further comprises a flexible member configured to move with the expandable member, and wherein the marker is attached to the flexible member.

11. The apparatus of claim 10 wherein the expandable member comprises a balloon and the flexible member comprises a sheath around the balloon.

12. The apparatus of claim 11 wherein the expandable member comprises a balloon and the flexible member comprises a mesh attached to the balloon.

13. A method of facilitating radiation treatment of a target in a patient, comprising:
positioning an expandable member in the patient with respect to the target;
expanding the expandable member to a desired size within the patient; and
determining a parameter the expandable member by localizing a marker that moves in association with movement of the expandable member.

14. The method of claim 13, further comprising inserting an ionizing radiation source into the expandable member and delivering ionizing radiation to the target.

15. The method of claim 14 wherein the expandable member comprises a balloon and the marker comprises a wireless transponder, and wherein localizing the wireless transponder comprises wirelessly transmitting an excitation energy to the marker, wirelessly transmitting a location signal from the marker in response to the excitation energy, and calculating a position of the marker in an external coordinate system based on the location signal.

16. The method of claim 15 wherein determining a parameter of the expandable member comprises determining whether the expandable member has changed from the desired size.

17. The method of claim 15 wherein determining a parameter of the expandable member comprises determining relative movement between the expandable member and a known reference location at the patient.

18. The method of claim 17, further comprising attaching a reference marker to the patient to define the known location, and wherein the reference marker comprises a second wireless transponder that wirelessly transmits a second location signal in response to a second wirelessly transmitted excitation energy.

19. The method of claim 14 wherein a plurality of wireless transponders are configured to move with the inflatable member, and wherein localizing the wireless transponders comprises (a) wirelessly transmitting individual location signals from individual wireless transponders in response wirelessly transmitted excitation energy and (b) calculating positions of the wireless transponders in an external coordinate system based on the location signals.

20. The method of claim 19 wherein determining a parameter of the expandable member comprises determining whether the expandable member has changed from the desired size.

21. The method of claim 19 wherein determining a parameter of the expandable member comprises determining relative movement between the expandable member and a known reference location at the patient.

22. The method of claim 21 wherein determining the relative movement between the expandable member and the known reference location occurs while delivering ionizing radiation to the target.

23. The method of claim 19 wherein determining a parameter of the expandable member comprises determining a rotational orientation of the expandable member within the patient.

24. The method of claim 14, further comprising determining a location of the ionizing radiation source by localizing another marker configured to move with the ionizing radiation source.
